(19) United States
(12) Patent Application Publication
(10) Pub. No.: US 2013/0090731 A1
(43) Pub. Date: Apr. 11, 2013

(54) Ligament Retainer Device and Method

(71) Applicant: Peter Michael Sutherland Walker, Bellevue Hill (AU)

(72) Inventor: Peter Michael Sutherland Walker, Bellevue Hill (AU)

(21) Appl. No.: 13/704,226

(22) PCT Filed: Jun. 22, 2011
(86) PCT No.: PCT/AU2011/000747

§ 371 Date: Dec. 13, 2012

(30) Foreign Application Priority Data
Jun. 22, 2010 (AU) 2010902703

(51) Int. Cl. A61F 2/08

(52) U.S. Cl. CPC A61F 2/0811
USPC 623/13.14

(57) ABSTRACT

A graft retainer device and method for substantially retaining a portion of a graft with respect to a bone of a patient. The device comprises a body; and a retaining element for retaining a graft with respect to the body. A surgical screw fastener device can be use with the graft retainer device for pulling a graft through a tunnel defined in the bone. The graft can includes any one or more of the set comprising: a transplant tendon, an artificial tendon, a transplant ligament, and an artificial ligament.

### FIELD OF THE INVENTION

The present invention relates to fastening devices and in particular to devices retaining grafts.

The invention has been developed primarily for use as a device that can be used to retain a graft (including tendon or ligament) within a bone and will be described hereinafter with reference to this application. However, it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

The present disclosure will be used with reference to an anterior cruciate ligament (“ACL”) reconstruction, but it will be understood that the technology and methods of the present invention may have other applications.

The ACL reconstruction can be done in numerous ways. All common methods involved drilling holes or tunnels in the femur and tibia. These can be drilled from any direction using a variety of techniques. Grafts such as autografts, allografts or artificial biomaterials may be used to extend between the femoral tunnel and the tibial tunnel. The graft is then fixed to the appropriate bone structure, again numerous techniques being suitable. The replacement graft is fixed to the femur and tibia, most commonly by a screw into the adjacent bone, it being understood that staples, pins and similar devices may also be used. In general, and most commonly, the graft is tensioned prior to finally affixing it to the bone. Devices are known that can exert tension onto the graft before affixation to the bone takes place. These tension devices hold the graft in tension while fixing screws are inserted to fasten the graft in place. Therefore, this tension device can not fine tune or adjust the tension of the graft once the remaining loose end is fixed in place.

### OBJECT OF THE INVENTION

It is an object of the present invention to overcome or ameliorate at least one of the disadvantages of the prior art, or to provide a useful alternative.

It is an object of the invention in its preferred form to provide a device for retaining a graft (including tendon or ligament) within a bone. Preferably, the device can retain the graft as it is tensioned during an ACL reconstruction.

It is an object of the invention in its preferred form to provide a device and methods for enabling adjustment of graft tension, after both ends of the graft are retained within a respective bone.

### SUMMARY OF THE INVENTION

According to an aspect of the invention there is provided a graft retainer device comprising:
a body; and
an retaining element for retaining a graft with respect to the body.

Preferably, the body defines an aperture for receiving a graft. Preferably, the element for retaining a graft with respect to the body is a gripping element for retaining a graft with respect to the body.

Preferably, the graft retainer device is adapted to abut the bone. More preferably, the body defines an abutment surface for abutting a bone. Most preferably, the abutment surface is typically provided by a circumferential flange. Alternatively, an interference fit can preferably be established between an abutment surface of the body and bone tunnel.

Preferably, the device is drawn at least partially into the bone to thereby minimise or reduce protrusion of the device above the surface of the bone.

Preferably, the device is fixed to the graft before being inserted into a bone tunnel, or prior to being drawn (or pulled) into position. More preferably, the device abuttingly engages the bone before the graft is tensioned.

Preferably, the gripping element includes a moveable inner gripping element. Alternatively, the gripping element preferably includes a woven suture element. Alternatively, the gripping element preferably includes a fluid agent inserted into the body.

Preferably, the gripping element comprises an insert component including a plurality of interconnected elements. More preferably, each resilient interconnected element has a respective finger protrusion. Most preferably, the resilient interconnected elements are interconnected about a circumference by resilient hinge members. Preferably, the resilient connection enables the finger protrusions to move radially inwardly and/or outwardly in use. The interconnected finger protrusions preferably define an aperture for receiving a graft.

Preferably, the body comprises two body elements. More preferably, the two body elements, when assembled, define the aperture for receiving a graft.

Preferably, gripping protrusions are included within the aperture.

Preferably, the device can be integrally formed with an artificial graft.

According to an aspect of the invention there is provided a surgical screw fastener device for pulling a graft through a first tunnel defined in a first bone, the surgical screw fastener including:
a body having a proximal end and a distal end;
an exterior screw thread located around the body for threadedly engaging a wall of the first tunnel;
a first coupling element at the proximal end of the body, the first coupling element adapted to couple a driver tool; and
a second coupling element for rotatable coupling a first end of the graft with respect to the screw fastener device.

Preferably, the passage is a through passage having an aperture at the distal end and the proximal end.

Preferably, the first coupling element is a female socket at the proximal end of the body. More preferably, the passage is a through passage having an aperture at the proximal end that defines the socket.

Preferably, the first coupling element is a releasable coupling element for securely coupling the screw fastener device to the driver tool.

Preferably, the passage has an aperture at the distal end; and the second coupling element comprises a saddle element that is locatable within the passage and is adapted to be rotatable with the passage. More preferably, the saddle element is locatable within the passage by being passed through an aperture at the proximal end defined by the passage. Most preferably, the saddle element is adapted to rotate freely within the passageway and is restrained in its axial movement toward the distal end by a necking down of the passageway.

Preferably, the second coupling element comprises a saddle element locatable within the passage, and is adapted to be rotatable with the passage; and the second coupling element further comprises a fastening element coupled to the saddle element and adapted to retain a first end of the graft. Preferably, the fastening element is integrally formed with the saddle element. Preferably, the fastening element is integrally formed with an artificial graft. Preferably, the fastening element is constructed of a flexible material.

Preferably, the fastening element includes any one or more of the set comprising: a loop element; a net element.

Preferably, selective clockwise or anticlockwise rotation of the body, while threadedly engaging the wall of the first tunnel, can respectively increase or decrease tension applied to the graft. Alternatively, selective anticlockwise or clockwise rotation of the body, while threadedly engaging the wall of the first tunnel, preferably respectively increase or decrease tension applied to the graft.

According to an aspect of the invention there is provided a method of using a surgical screw fastener device for pulling a graft through a first tunnel defined in a first bone, the method comprising the steps of:
(a) providing a screw fastener device;
(b) coupling the screw fastener to a driver tool passed through the first tunnel;
(c) rotatably coupling a first end of the graft to the screw fastener device;
(d) rotating the screw fastener, by rotating the driver tool, causing the screw fastener to threadedly engage the bone and thereby draw the graft up through the tunnel;
(e) with the other end of the graft fixed in location, the screw fastener can be rotated with respect to the bone to thereby set a tension applied to the graft; and
(f) detaching the screw fastener from the driver tool.

According to an aspect of the invention there is provided a method of substantially retaining a portion of a graft with respect to a bone of a patient, while using a surgical screw fastener to pull the graft through a first tunnel defined in the bone, the method comprising the steps of:
(a) providing a graft retainer device;
(b) fixing the graft retainer device to a first portion of the graft;
(c) providing a screw fastener;
(d) coupling the screw fastener to a driver tool passed through the first tunnel;
(e) rotatably coupling a second portion of the graft to the screw fastener;
(f) rotating the screw fastener, by rotating the driver tool, causing the screw fastener to threadedly engage the bone and thereby draw the graft up through the tunnel;
(g) with the first portion of the graft retained, the screw fastener can be rotated with respect to the bone to thereby set a tension applied to the graft; and
(h) detaching the driver tool from the screw fastener.

Preferably, the screw fastener device is as herein described.

Preferably, fixing the other end of the graft includes abutment of an end plug fixed to the other end of the graft as a result of the screw fastener device drawing up the graft.

According to an aspect of the invention there is provided a screw fastener having a longitudinal through passageway. One end of the passageway comprises a socket for receiving a fastener driver tool (or adaptor). The other end of the passageway provides a portal for the looped material or artificial graft.

A saddle is preferably located within the passageway. The saddle is not able to pass through the loop portal. The saddle is adapted to receive a suture, suture loop or a loop of material through which the graft is placed. More preferably, the saddle is able to rotate within the passageway when the suture and/or loop is in tension.

A saddle is preferably located within the passage but not able to pass through the loop portal. More preferably the saddle can rotate within the passage. Most preferably, with the screw fastener located within a tunnel (or hole) formed in a bone, the saddle enables the screw fastener to be rotated with respect to the bone without the tendon undergoing a corresponding rotation.

Preferably a screw fastener can be located within a tunnel (or hole) formed in a bone and rotatably coupled to a graft, wherein rotation of the screw fastener with respect to the bone pulls the graft through the tunnel. More preferably, rotation of screw fastener enables controlled pulling of a graft up through a tunnel. Most preferably, with each end of the graft coupled to a respective bone, rotation of screw fastener in one or another direction enables a respective increased and decreased tensioning of the graft.

In preferred embodiments the fastener has a pair of transverse openings, each leading to a longitudinal, external channel. The transverse openings in the channels are preferably adapted to receive a fastener such that the driver can be rotated in either direction. A lip on the inside of the screw prevents it from being pulled out of the screw. An alternative technique to couple the driver to the screw would be to have holes and a form of attaching suture to the screw for tying it to the driver either in a slot on the side of the driver or through the middle of the driver if cannulated.

A screw fastener preferably includes a locking mechanism that can pull the screw, in tension, thus into place and then be unlocked once the screw has the correct tension.

Preferably the driver is adapted to provide an indication of the torque on the fastener.

Preferably, the screw fastener can be pulled into position by a driver. More preferably, the driver can be released once the screw fastener is in position.

Preferably a graft includes any one or more of the set comprising: a transplant tendon, an artificial tendon, a transplant ligament, and an artificial ligament.

Preferably, a screw faster device is used in combination with a graft retainer device, as described herein.

### BRIEF DESCRIPTION OF THE DRAWING FIGURES

In order that the invention be better understood, reference is now made to the following drawing figures in which:

FIG. 1A is an underside perspective view of an embodiment fastener made in accordance with the teachings of the present invention;

FIG. 1B is a top perspective view of the fastener depicted in FIG. 1A;

FIG. 1C is a side elevation of the fastener depicted in FIG. 1A;

FIG. 1D is a cross section through line A-A of FIG. 1C;

FIG. 1E is a perspective view of a saddle, according to FIG. 1D;

FIG. 1F is a side elevation view of an embodiment fastener with the saddle retaining a suture loop that passes through the portal;

FIG. 1G is a cross sectional view through line B-B of FIG. 1F;

FIG. 2A is a perspective view of another embodiment form of driver and driver engagement;

FIG. 2B is an enlarged perspective view of FIG. 2A, showing detail of the driver engagement;

FIG. 2C is a cross sectional view of an embodiment driver and engagement of FIG. 2A;

FIG. 2D is a side elevation of the device depicted in FIG. 2A;

FIG. 3A is a perspective view of a further embodiment of a driver;

FIG. 3B is a cross section and detail of the driver depicted in FIG. 3A, illustrating the engagement;

FIG. 3C is an enlarged cross section of FIG. 3B, showing detail of the driver engagement;

FIG. 4A is a perspective view of an embodiment screw fastener made in accordance with the teachings of the present invention;

FIG. 4B is a sectional view of the screw fastener of FIG. 4A;

FIG. 5A is a perspective view of an embodiment screw fastener made in accordance with the teachings of the present invention;

FIG. 5B is a sectional view of the screw fastener of FIG. 5A;

FIG. 6A is a sectional view of an embodiment fastener made in accordance with the teachings of the present invention;

FIG. 6B is a sectional view of an embodiment fastener made in accordance with the teachings of the present invention;

FIG. 6C is a sectional view of an embodiment fastener made in accordance with the teachings of the present invention;

FIG. 6D is a perspective view of an embodiment fastener made in accordance with the teachings of the present invention;

FIG. 7A-7K are perspective views illustrating steps involved in utilisation of the invention in conjunction with an ACL reconstruction;

FIG. 8A-8D are perspective views illustrating steps involved in utilisation of the invention in conjunction with an ACL reconstruction;

FIG. 9 is a flowchart for an embodiment method of a screw fastener in conjunction with an ACL reconstruction;

FIG. 10A-10C are perspective views illustrating steps involved in utilisation of an embodiment graft retainer device accordance with the teachings of the present invention in conjunction with an ACL reconstruction;

FIG. 11A is a perspective view of an embodiment graft retainer device made in accordance with the teachings of the present invention;

FIG. 11B is a sectional view of the graft retainer device of FIG. 11A;

FIG. 12A is a perspective view of an embodiment graft retainer device made in accordance with the teachings of the present invention;

FIG. 12B is a perspective view of the graft retainer device of FIG. 12A, shown in tension;

FIG. 13A is a perspective view of the graft retainer device of FIG. 12A, shown in use;

FIG. 13B is a perspective view of the graft retainer device of FIG. 12A, shown in use;

FIG. 14A is a perspective view of an embodiment graft retainer device made in accordance with the teachings of the present invention;

FIG. 14B is a perspective view of the graft retainer device of FIG. 14A, shown retaining a graft;

FIG. 15A is a perspective view of an embodiment graft retainer device made in accordance with the teachings of the present invention;

FIG. 16A is a perspective view of an embodiment graft retainer device made in accordance with the teachings of the present invention;

FIG. 16B is a plan view of the graft retainer device of FIG. 16A;

FIG. 16C is a side elevation view of the graft retainer device of FIG. 16A;

FIG. 16D is a sectional view of the graft retainer device of FIG. 16A;

FIG. 17A is a plan view of a component element of a embodiment graft retainer device made in accordance with the teachings of the present invention;

FIG. 17B is a side elevation view of the graft retainer device of FIG. 17A;

FIG. 17C is a sectional plan view of the graft retainer device of FIG. 17A;

FIG. 17D is a plan view of the graft retainer device of FIG. 17A, showing two co-operating component elements;

FIG. 17E is a perspective view of a graft retainer device, showing two co-operating component elements in closed clamping engagement;

FIG. 17F is a perspective view of a graft retainer device, showing two co-operating component elements in clamping engagement;

FIG. 17G-17J are perspective views component elements of a embodiment graft retainer device according to FIG. 17E;

FIG. 18A a perspective view of an embodiment graft retainer device made in accordance with the teachings of the present invention, shown being coupled to a graft;

FIG. 18B is a perspective view of the graft retainer device of FIG. 18A, shown retaining a graft;

FIG. 19A a perspective view of an embodiment graft retainer device made in accordance with the teachings of the present invention, shown being coupled to a plurality of grafts;

FIG. 19B is a perspective view of the graft retainer device of FIG. 19A, shown retaining a plurality of grafts.

### BEST MODE AND OTHER EMBODIMENTS

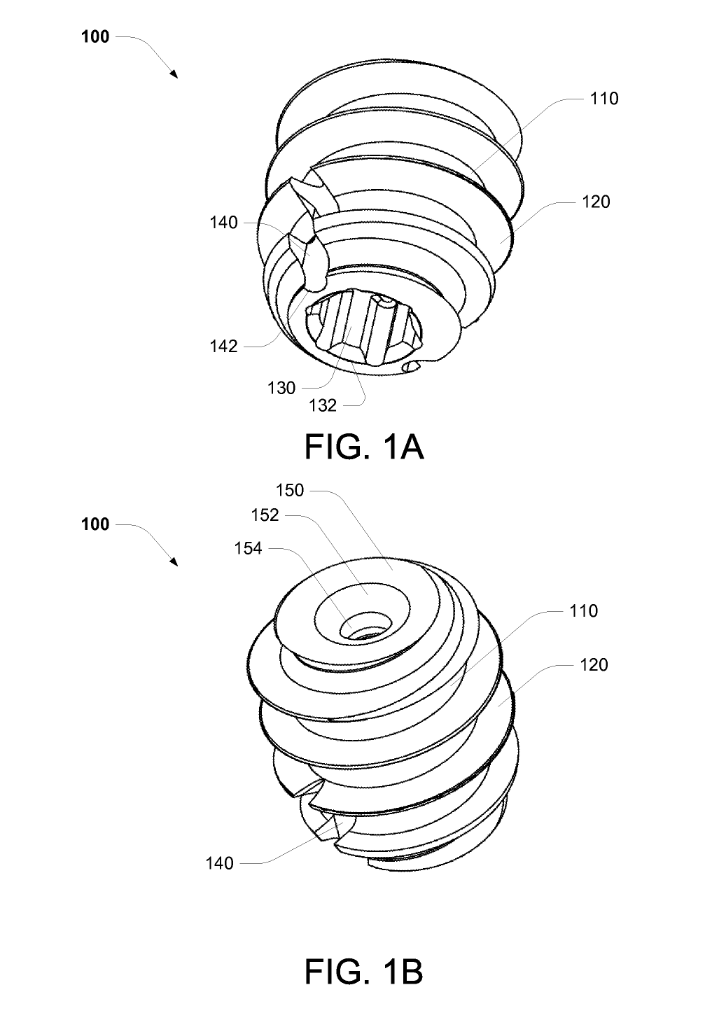

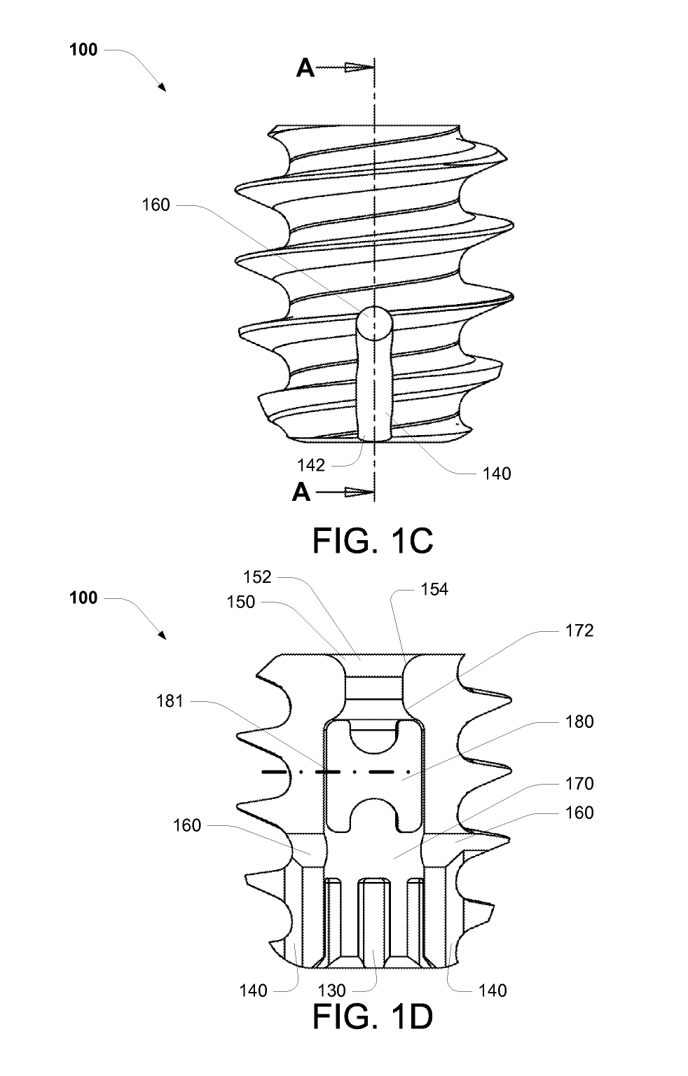

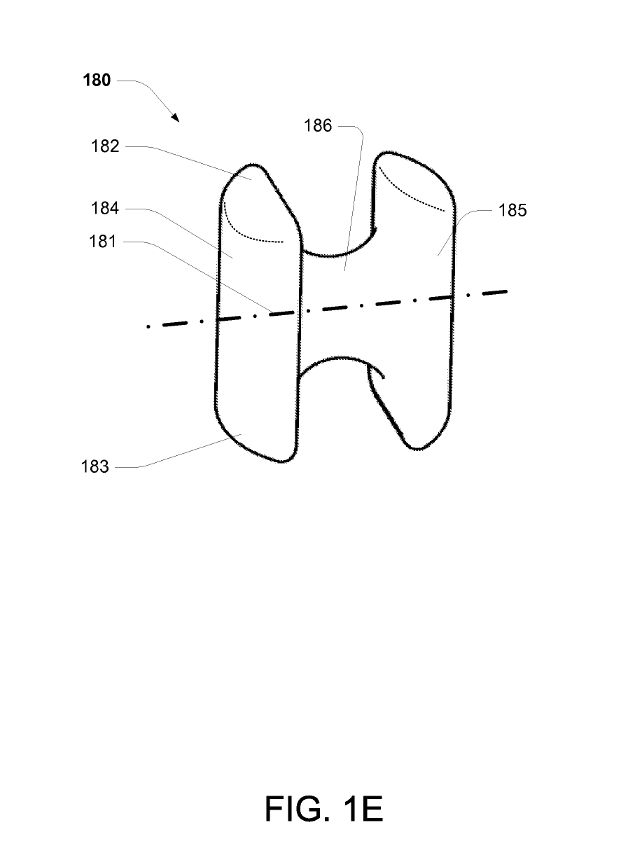

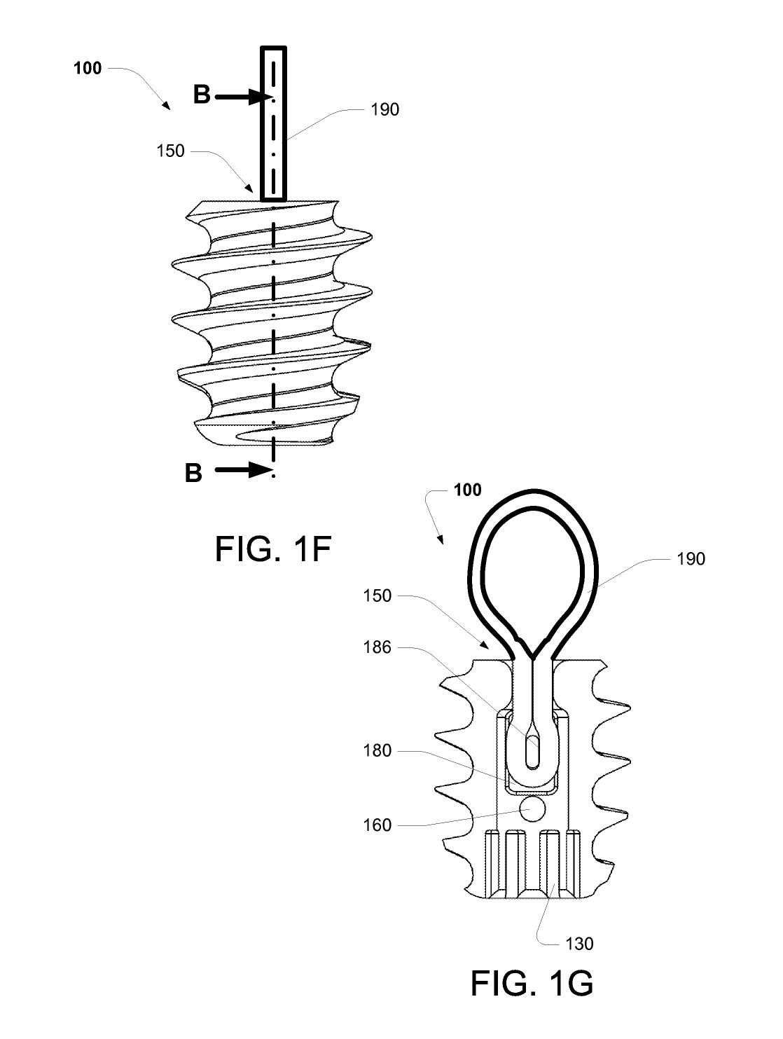

As shown in FIG. 1A, a fastener 100, in accordance with the teachings of the present invention, comprises a relatively coarse threaded slightly tapered plug (or body) 110 having the fastening characteristics of a bone screw. External screw threads 120 are adapted to be self tapping into a tunnel pre-drilled through the tibia or femur. A central longitudinal bore or passageway 130 extends through the fastener, from one end to the other.

By way of example, a device of this kind will typically be about 6 mm to 14 mm in diameter and have a length of about 15 mm to 20 mm.

In an embodiment, a proximal end of the passageway 132 forms a socket for receiving a driver (including an adaptor and/or tool) such as a Torx brand driver. It will be appreciated that the socket, and therefore the head of a corresponding driver, can comprise numerous configurations, including a hex socket, and/or a star socket. A driver is adapted to accommodate the socket configuration.

Opposing longitudinal channels 140 extend approximately a third to halfway down the body of the fastener, into the screw threads, providing a relief groove that starts by intersection of the proximal rim 142 of the fastener and terminates at one of a pair of transverse through openings (not shown in this view). The channel 140 interrupts the screw threads and the proximal rim providing a space that can accommodate a loop of material such as polyethylene or polyester or other type of suture material without interfering with the operation of the fastener, the fastener's threads or the socket 130. In this embodiment channels 140 are provided to enable the screw to facilitate more aggressive cutting engagement with the bone.

As shown in FIG. 1B, the distal end 150 of the fastener 100 comprises a portal 152 that leads into the central bore or through passageway 130. The portal 152 comprises a smoothly radiused rim and a smooth opening 154 for receiving the flexible loop or suture arrangement that will be described with reference to FIG. 1F and FIG. 1G.

As shown in FIG. 1C and FIG. 1D, each longitudinal, external channel 140 terminates in a transverse through opening. The through openings lead into the central passageway 170. The longitudinal channel and transverse channels can be used for engaging and/or locking a cooperating pulling device or driver.

A saddle 180 is adapted to rotate freely within the passageway and is restrained in its axial movement toward the portal 152 by a necking down 172 of the passageway adjacent to the portal 152. The saddle is not able to pass through the loop portal due to a lip on the inside of the screw fastener. The saddle rotates within the screw as a graft is being pulled up so as to not twist the graft (or tendon or ligament).

It will be appreciated that, references to a graft includes a transplant or artificial tendons and/or ligaments.

As shown in FIG. 1D, the central passageway 170 is adapted to receive a saddle 180 between the openings 160 and the necking 172. In preferred embodiments, the saddle is symmetrical about its transverse axis 181 so that it may be inserted into the passageway and used in either orientation. The edges of the longitudinal ends 182, 183 are radiused to cooperate with the necking 172, thus reducing friction. Note that the through openings 160 are formed beyond the axial reach of the socket 130 so that the suture that passes through the openings 160 does not interfere with the head of the driver.

As shown in FIG. 1E, the saddle 180 is generally “H” shaped, but can be of other shapes. The lateral components 184, 185 are essentially sections of cylinder and are joined together by a smooth integral cross member 186. The cross member 186 is smoothly blended into the interior surfaces of the lateral portions 184, 185. The cross member 186 is necked, providing a minimum diameter in the middle and a gradual flaring toward the lateral members 184, 185.

As shown in FIG. 1F and FIG. 1G, a loop of fibre material (for example suture material) 190 may be passed around the cross member 186 of the saddle 180 to form a constrained loop. The loop 190 enters through and exits through the loop portal 152. Note the lack of sharp edges in the area of the portal.

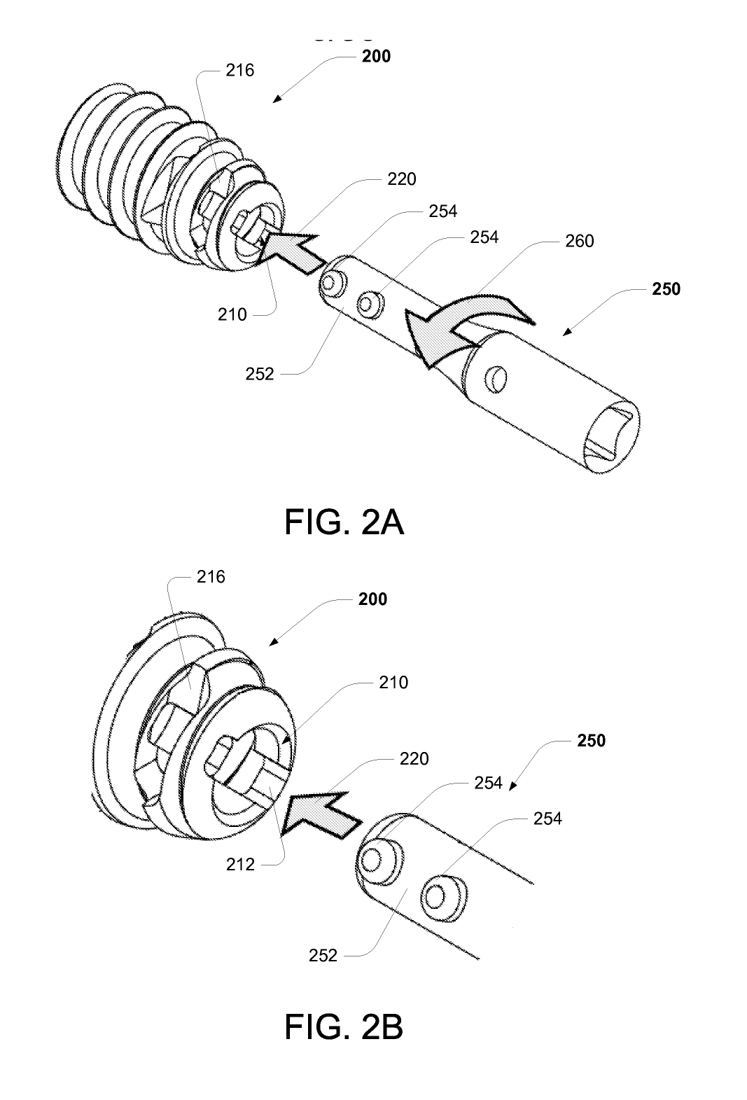

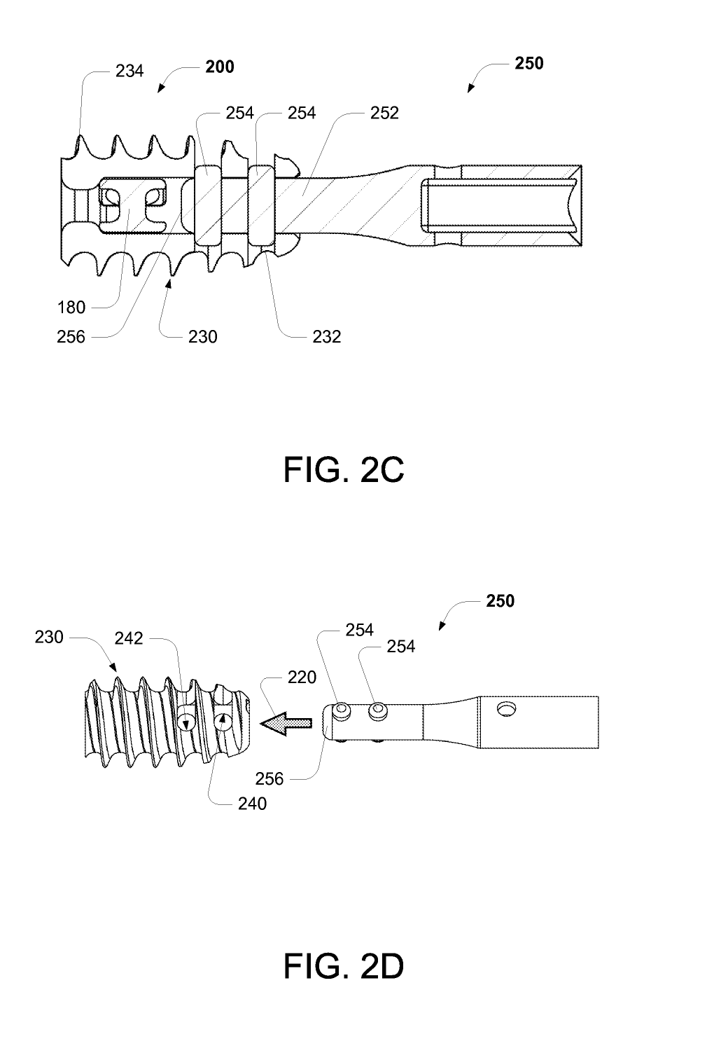

As shown in FIG. 2A and FIG. 2B a fastener 200 has been configured to receive a specially adapted driver 250. The driver 250 comprises a generally cylindrical tip 252 having one or more radially extending pins or projections 254. In this example, the driver 250 is provided with four pins. The pins are provided in adjacent pairs that are diametrically opposed to one another on the tip 252. It will be appreciated that other configurations are contemplated. In particular, in an embodiment only one pin or protrusion may be provided. It will be appreciated that pins can be of any cross section, for example circular, square or rectangular.

In order that the fastener 200 receive the tip of the driver 252, the internal bore 210 of the fastener 200 is provided with a pair of opposing internal longitudinal grooves 212. The area radially outward of the terminus of a groove is machined away 216. Clockwise rotation of the driver causes the pins 254 to abut an adjacent portion of the fastener 200 and thus cause the fastener to rotate and advance in the forward direction 220. However, counter clockwise rotation of the driver 260 causes the pins 254 to rotate and thus depart from the groove or grooves 212 and come to rest in a position where withdrawal of the driver tip 252 is resisted by a portion of the fastener body. In this orientation, anti-clockwise rotation of the driver 260 acts to withdraw the fastener 200 (retrograde motion, i.e. in the direction opposite of arrow 220). Further, putting the driver into tension to assist in the withdrawal cannot disengage the driver from the fastener 200. A small clockwise rotation of the driver realigns the pins 254 with the channels 212 so that the tip 252 can be withdrawn from the fastener 200.

As shown in FIG. 2C, the driver tip 252 can be constructed by providing transverse passageways for receiving the pins 254. In this example, two pins extend through the entire diameter of the driver and beyond the outer surface to create four projections 254. Note that the configuration of the internal grooves 212 prevents the extreme distal tip 256 of the driver from making contact with the saddle 180. FIG. 2C also illustrates that by way of example only, and according to the present embodiment the radial extent of the pins 254 is below the root 232 of the cutting threads 230. By making the tip diameter of the pins 254 smaller than even the smallest diameter root 234, the insertion of the driver and its pins 254 is never resisted by bone material that may occupy the space between the threads 230.

As shown in FIG. 2D each pin 254 comes to rest, after the driver has been inserted and rotated counter clockwise into a transverse side channel 240. In the side channel (withdrawal position) it is preferred that the round pins 254 make surface contact 242 with the body of the fastener 200. This requires that the side walls 242 of the groove's side-channel have a generally semi circular configuration where they are contacted by a round pin. It will be appreciated that, in an alternative embodiments, other co-operating configurations can be used, for example substantially rectangular pins and a square set groove.

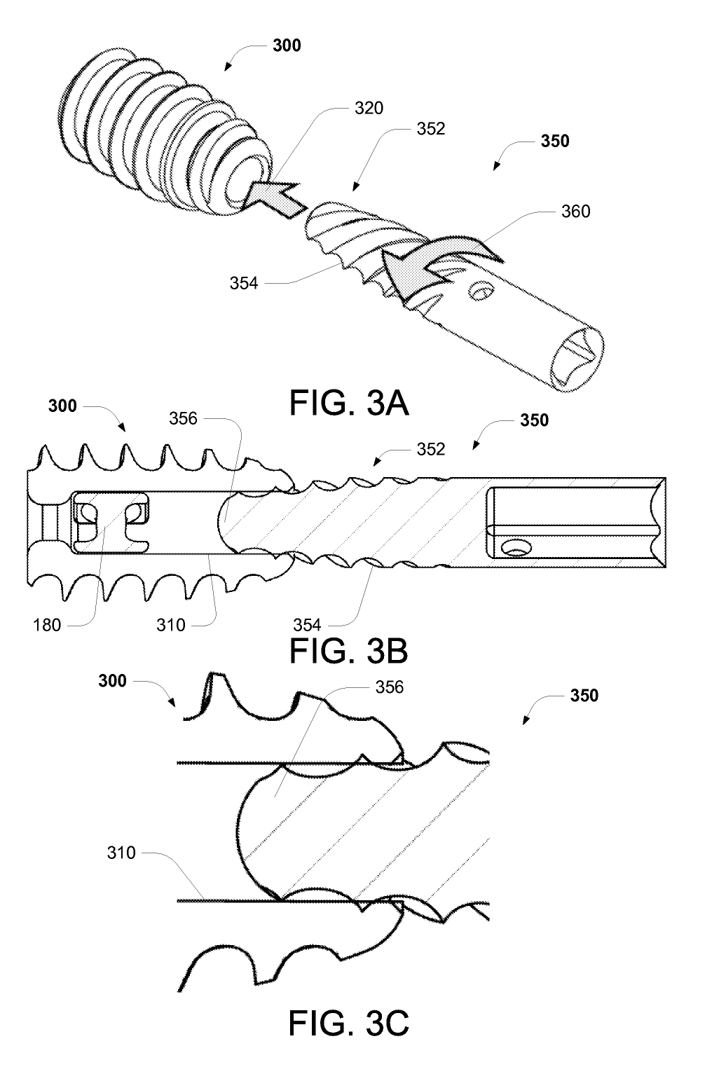

Another example of a driver is depicted in FIG. 3A and FIG. 3B. A retrograde fastener 300 has been configured to receive a specially adapted driver 350. In this example, the tip 352 of the driver is in the form of a fastener extractor. The tip has hardened, tapered, coarse threads 354 that are anti-clockwise. As shown in FIG. 3B and FIG. 3C, the extreme tip 356 of the driver can be inserted into the smooth interior bore 310 of the fastener 300. Anti-clockwise rotation causes the threads 354 to advance and cut into the bore 310. The fastener 300 is thus withdrawn through the bone tunnel with the anti-clockwise motion of the driver. The driver can be put into considerable tension without the threads 354 disengaging. Clockwise rotation of the driver causes the tip 356 to reverse out of the bore 310 and thus causes disengagement of the driver with the fastener 300. It will be appreciated that for this type of driver, the threads 354 must be harder than the internal bore 310 of the fastener 300.

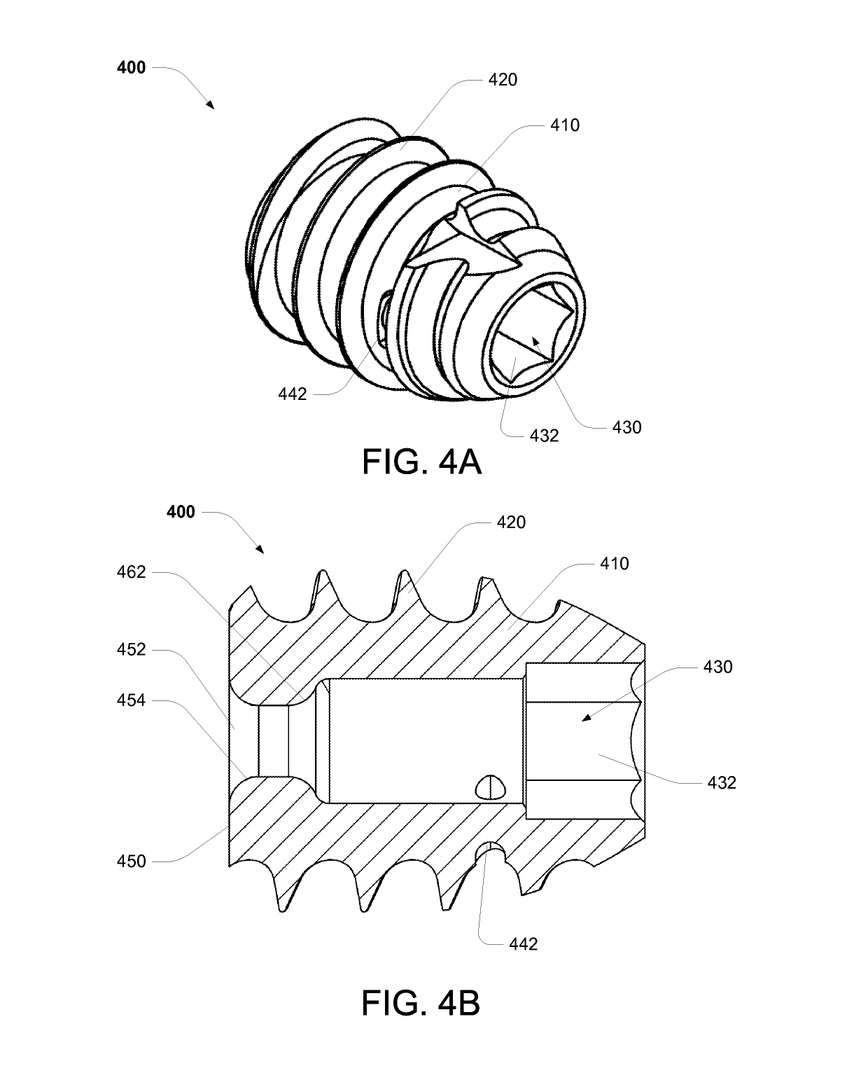

FIG. 4A and FIG. 4B show an embodiment screw fastener 400. A first coupling element is shown at the proximal end of the body for coupling a driver tool.

This embodiment (similar to the embodiment screw fastener 100) comprises a relatively coarse threaded slightly tapered plug (or body) 410 having the fastening characteristics of a bone screw. External screw threads 420 are adapted to be self tapping into a tunnel pre-drilled through the tibia or femur. A central longitudinal bore or passageway 430 extends through the fastener, from one end to the other. The proximal end 432 of the passageway forms a socket for receiving a cooperating driver. It will be appreciated that the socket, and therefore the head of a corresponding driver, can comprise numerous configurations, including an inwardly scalloped hex socket. A receiving driver is adapted to accommodate the socket configuration. One or more transverse through openings 442 can accommodate a loop of material such as polyethylene or polyester or other type of suture material without interfering with the operation of the fastener, the fastener's threads or the socket 430.

As shown in FIG. 4B, the distal end 450 of the fastener 400 comprises a portal 452 that leads into the central bore or through passageway 430. The portal 452 comprises a smoothly radiused rim and a smooth opening 454 for receiving the loop or suture arrangement as herein described. A saddle (not shown) is adapted to rotate freely within the passageway and is restrained in its axial movement toward the portal 452 by a necking down 462 of the passageway adjacent to the portal 452.

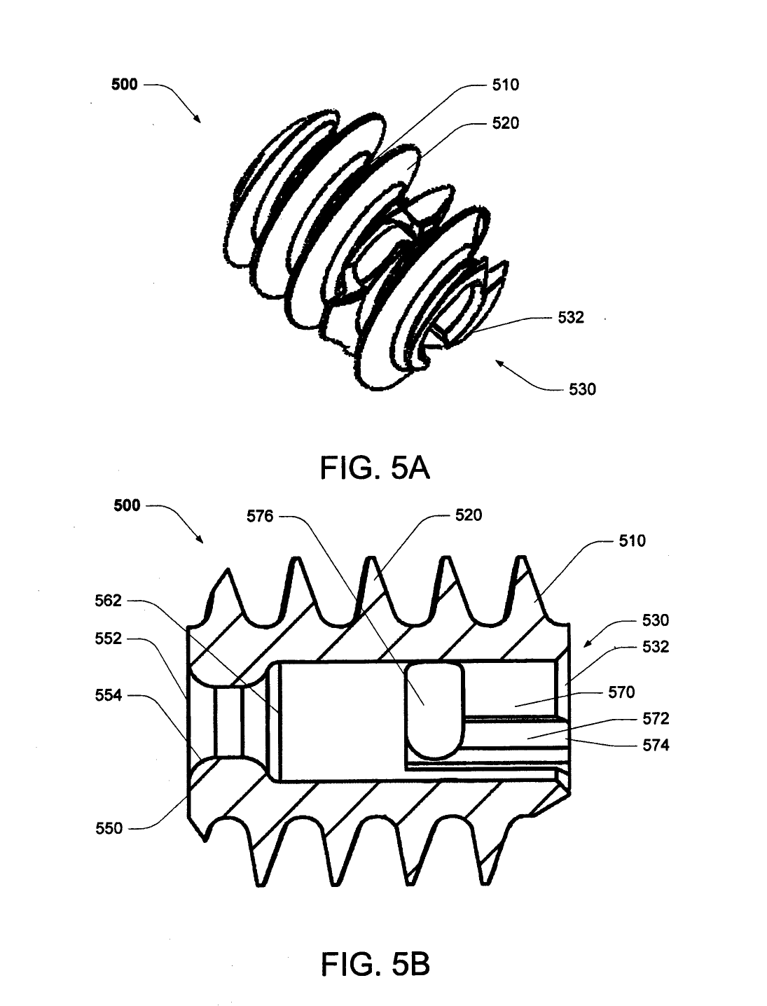

FIG. 5A and FIG. 5B show an embodiment screw fastener 500. A first coupling element is shown at the proximal end of the body for coupling a driver tool.

This embodiment (similar to the embodiment screw fastener 100) comprises a relatively coarse threaded slightly tapered plug (or body) 510 having the fastening characteristics of a bone screw. External screw threads 520 are adapted to be self tapping into a tunnel pre-drilled through the tibia or femur. A central longitudinal bore or passageway 530 extends through the fastener, from one end to the other. The proximal end 532 of the passageway forms a socket for receiving a cooperating driver.

As shown in FIG. 5B, the distal end 550 of the fastener 500 comprises a portal 552 that leads into the central bore or through passageway 530. The portal 552 comprises a smoothly radiused rim and a smooth opening 554 for receiving the loop or suture arrangement as herein described. A saddle (not shown) is adapted to rotate freely within the passageway and is restrained in its axial movement toward the portal 552 by a necking down 562 of the passageway adjacent to the portal 552.

It will be appreciated that the socket, and therefore the head of a corresponding driver, can comprise numerous configurations, including a bayonet style connection. A receiving driver is adapted to accommodate the socket configuration.

In this embodiment, a bayonet style connection 570 can comprise one or more longitudinal channels 572 extending approximately a third to halfway down the periphery of longitudinal bore or passageway 530, providing a relief groove that starts by intersection of the proximal rim 574 of the fastener and terminates at a radially scribed passageway 576.

By way of example only, when inserting a driver (not shown) into the socket 530, the bayonet style connection 570 enables a releasable coupling such that the screw fastener 500 can be pulled through or to a tunnel in a bone. A pin on the driver engages and traverses the longitudinal channel 572, such that upon full insertion of the driver, the driver can be axially rotated such that the pin sweeps the radially scribed passageway 576.

The configuration of the internal channel (or grooves) are adapted to prevent the extreme distal tip of the driver from making contact with the saddle.

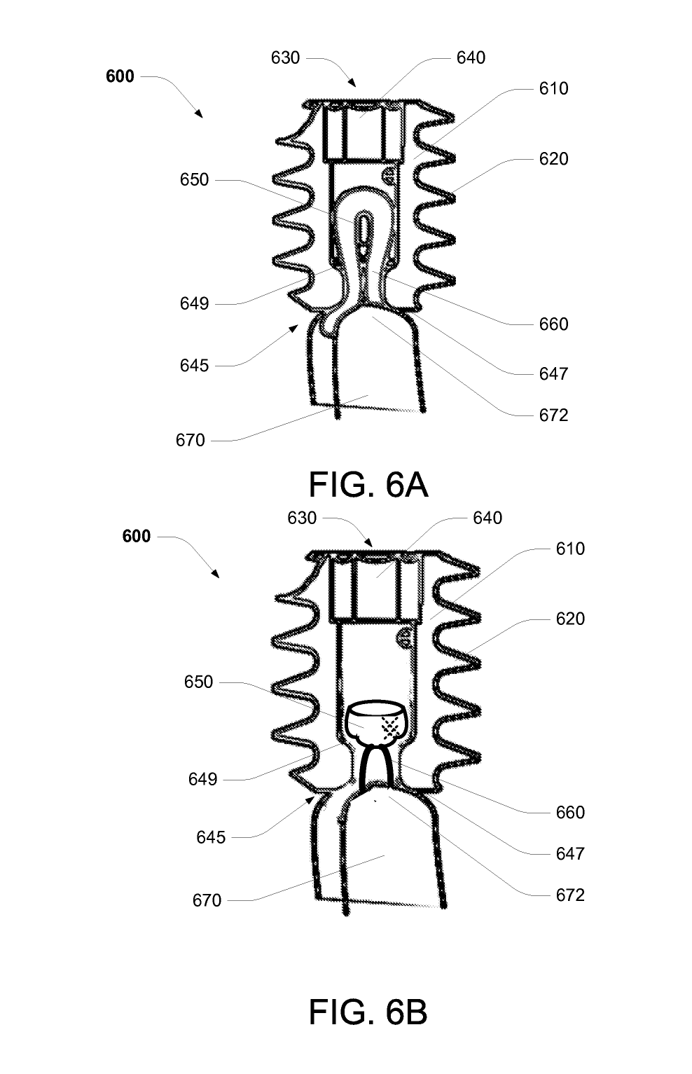

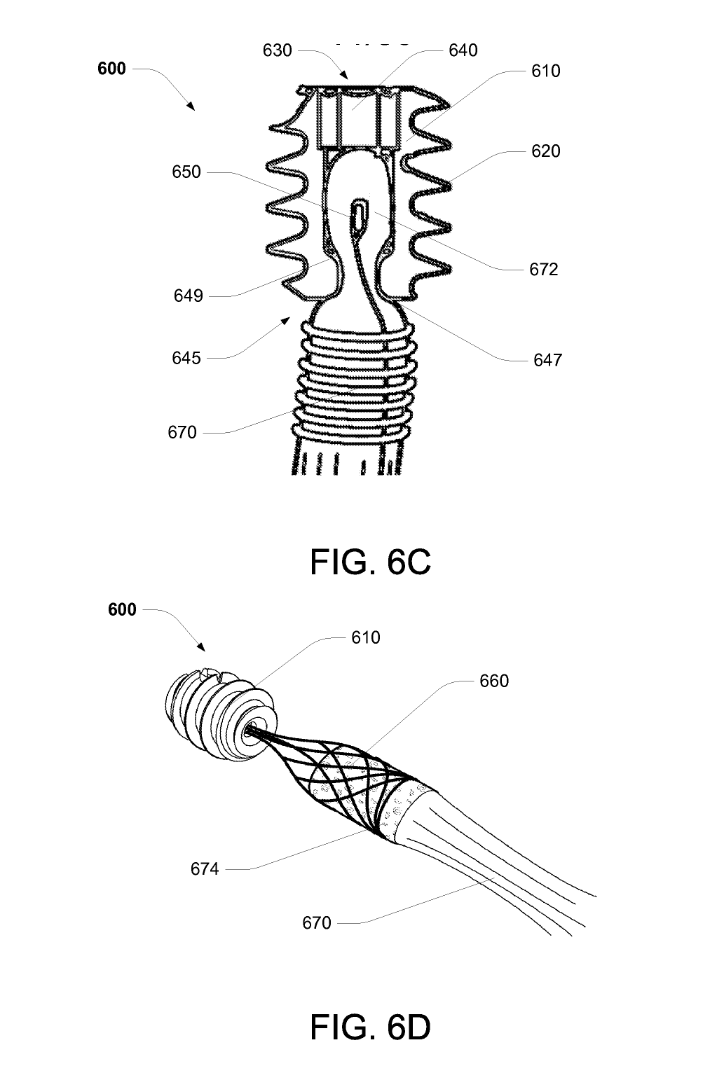

FIG. 6A through FIG. 6D show alternative structures fastening element for rotatably coupling (or attaching) a graft/tendons to a screw fastener. However, it will be appreciated that structures for rotatably coupling (or attaching) a graft are not limited to these particular embodiments. These embodiments shows alternative second coupling element for rotatable coupling of a first end of the graft with respect to the device.

The screw fastener includes a body 610 having an external screw thread 620. A central longitudinal bore or passageway 630 extends through the fastener, from one end to the other. The proximal end 640 of the passageway forms a socket for receiving a cooperating driver. The distal end 645 of the screw fastener comprises a portal 647 that leads into the central passageway 630. A saddle 650 is adapted to rotate freely within the passageway and is restrained in its axial movement toward the portal 647 by a necking down 649 of the passageway adjacent to the portal. The graft 670 is rotatably couplable to the screw fastener.

It will be appreciated that a first end 672 of replacement tendons or graft 670 will be rotatably couplable to a screw fastener, and the second (other) end fixedly couplable to a bone. The replacement tendons are provided by tendons that are looped around a coupling operatively associated with the screw fastener, the first end is defined by the portion of tendons/graft adjacent the screw fastener (when rotatably coupled) and the second end is defined by free ends of tendons/graft (or the other end).

FIG. 6A is a sectional view of an embodiment fastener 600 made in accordance with the teachings of the present invention.

In this embodiment, a flexible loop element 660 is located around the saddle 650. The loop extends below the portal 647, such that the graft (or tendons) can pass through and/or be coupled to the loop. The free ends of graft (or tendons) define the second end of the graft. A saddle 650 is adapted to rotate freely within the passageway thereby providing a rotatable coupling between the screw fastener and the graft 670. The saddle 650 and loop element 660 can be located in the passageway 630 by passing them through the opening at the proximal end 640.

FIG. 6B is a sectional view of an embodiment fastener made in accordance with the teachings of the present invention.

In this embodiment, a flexible loop 660 is integrally formed with the saddle 650, for example in the form of an expansion of the looped material. The loop extends below the portal 647, such that the graft (or tendons) can pass through and/or be coupled to the loop. The free ends of tendons define the second end of the graft. A saddle 650 is adapted to rotate freely within the passageway thereby providing a rotatable coupling between the screw fastener and the graft 670. The saddle 650 and loop element 660 can be located in the passageway 630 by passing them through the opening at the proximal end 640.

By way of example only, the combination saddle and loop can comprise a loop having an expanded substantially non-compressible end, such that the material and configuration were sufficiently non-compressible so that the expanded end would not pass through the necking down 649 of the passageway 630.

By way of example only, an expansion of the looped material can be in the form of a tight weave, a specialised knot or treatment of the loop material in such a way that it is restricted from passing through the necking down 649 of the passageway 630, but still enabled to rotate within the passageway 630

FIG. 6C is a sectional view of an embodiment fastener made in accordance with the teachings of the present invention.

In this embodiment, the graft 670 is received by the portal 647 and extended around the saddle 650. The free ends of tendons define the second end of the graft. A saddle 650 is adapted to rotate freely within the passageway thereby providing a rotatable coupling between the screw fastener and the graft 670. The saddle 650 and tendons/graft 670 can be located in the passageway 630 by passing them through the opening at the proximal end 640.

By way of example an artificial graft can be combined from multiple artificial tendon strands that extend around the saddle and are braided in situ, for providing a rotatable coupling to the screw fastener.

FIG. 6D is a sectional view of an embodiment fastener made in accordance with the teachings of the present invention.

In this embodiment, when performing a bone ligament reconstruction, a webbing configuration, grabbing suture configuration, trap type configuration, or the like 660 can be used to couple bone 672 at the first end of the transplanted tendon/graft 670. The configuration can be extended around the saddle, thereby providing a rotatable coupling to the screw fastener

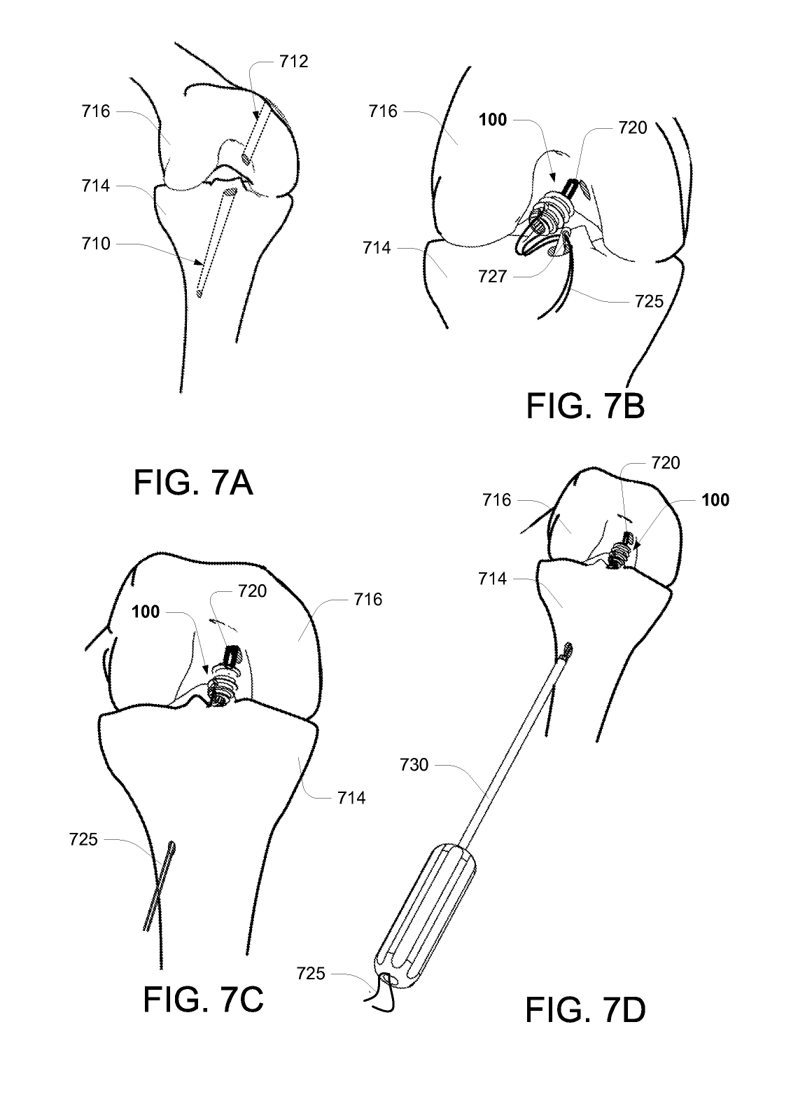

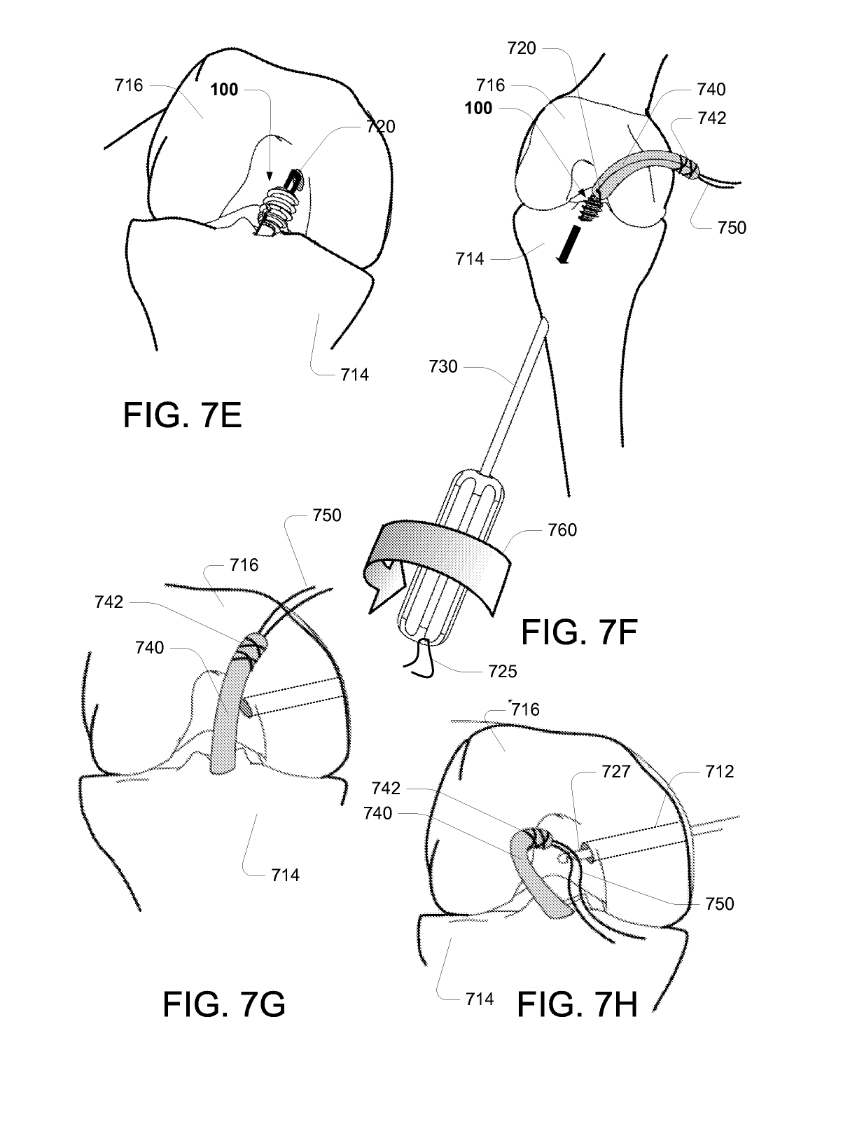

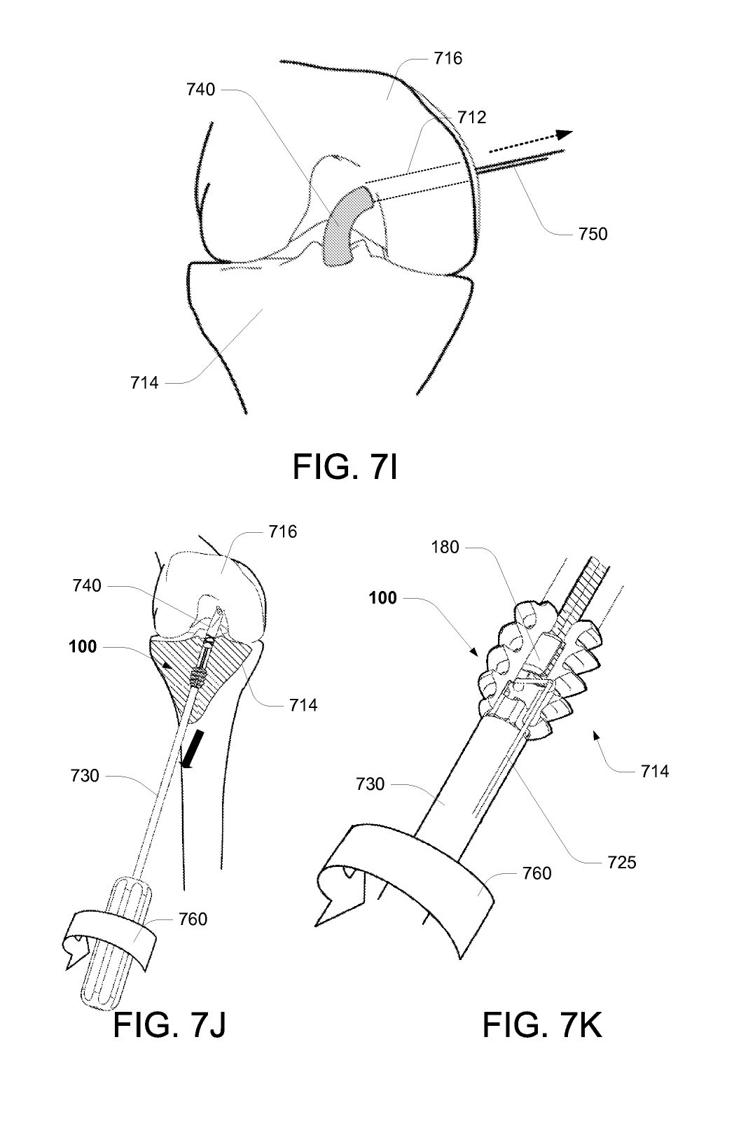

FIG. 7A-FIG. 7K illustrate how the fastener is used in an embodiment method of an ACL reconstruction method. As shown in FIG. 7A, through holes or tunnels 710, 712 are formed through the bones of the tibia 714 and the femur 716. As shown in FIG. 7B a first suture material 720 forms a loop around the saddle as illustrated in FIG. 6. Typically, the first suture material comprises a loop extending around the saddle and adapted to extend below the screw fastener for receiving the graft. A second suture 725 passes through the transverse openings 160. The second suture 725 is collected with a small hook 727 that is inserted through the tibial tunnel 710. The second suture is then withdrawn from the opposite end of the tibial tunnel. In one embodiment, the free end of the second suture 725 is passed e.g. through the head, shaft and handle of an appropriate driver 730, and tightened to the driver. This allows the driver 730 to be advanced through the tibial tunnel 710. As shown in FIG. 7F, the replacement tendon 740 is passed through the loop, whipped or otherwise attached to the first suture material 720. It will be appreciated that the tendon can be placed through the loop before it is inserted into the knee or could be coupled when the tendon is in the knee By way of example, first suture comprises a loop extending around the saddle and adapted to form a loop below the screw fastener for receiving the tendon therethrough. The driver 730 is fully inserted into the socket 130. In this position, the second suture 725 is used to attach or temporarily lock the fastener onto the head of the driver 730 by tensioning the free end of the second suture 725. As shown in FIG. 7H a third suture 750 is whip stitched onto the femoral side 742 of the replacement tendon structure 740 and the third suture 750 is picked up with a hook 727 and drawn through the femoral tunnel 712. As shown in FIG. 7I, the femoral end whip stitched suture is then pulled through the femoral tunnel and then affixed with an appropriate device to the femur bone. As suggested by FIG. 7J, the femoral end of the tendon is now fixed to the femur and the tibial end of the tendon is affixed to the saddle within the threaded fastener 100. At this point the driver 730 is rotated anti-clockwise 760, thus retracting the fastener 100 into the tunnel 710 toward the driver 730. It will be appreciated that in an alternative embodiment the screw thread can be configured such that clockwise rotation of the driver causes the screw fastener to be retracted into the tunnel. The retraction of the fastener 100 tensions the tendon and the degree of tension is determined by the extent of rotation and/or torque imposed by the surgeon. Because the saddle 180 rotates freely within the fastener 100, the tendon does not become twisted as it becomes tensioned. When the appropriate tension is reached, the driver 730 and the second suture 725 can be withdrawn from the tibial tunnel 710. This procedure can be done in either direction such that the screw can end up in the femur or tibia.

In an embodiment, a suture is used to fasten a driver to a screw fastener. The suture can be attached to the screw, typically passing through 2 holes in the screw fastener. By way of example, the suture can then be located to the side of the driver, or pass through the middle of a cannulated driver. The suture can be tensioned and tied at the proximal end of the driver to hold the screw in place so it can be pulled/drawn up to a tunnel in a bone. The suture is typically removed once the screw is in placed.

An embodiment driver (or adaptor), not shown, is used to locate a screw fastener in a bone (for example screw fastener 500).

This embodiment driver typically has an elongate shaft, terminating at one end with a coupling element for engaging a socket of a screw fastener.

An embodiment driver can comprise a generally cylindrical tip (distal tip) having one or more radially extending pins or projections. For example, the driver can be provided with two oppositely directed radially extending pins on the tip. It will be appreciated that other configurations are contemplated. In particular, in an embodiment only one pin or protrusion is provided.

In order that the fastener (for example screw fastener 500, not shown) receive the tip of the driver, the internal bore of the fastener is provided with a pair of opposing internal longitudinal channels or grooves. The area radially outward of the terminus of a groove is machined away. Clockwise rotation of the driver causes the pins to abut an adjacent portion of the fastener and thus cause the fastener to rotate and advance in the forward direction. Counter clockwise rotation of the driver causes the pins to rotate and thus depart from the longitudinal channel or groove and come to rest in a position where withdrawal of the driver tip is resisted by a portion of the fastener body. In this orientation, further anti-clockwise rotation of the driver acts to draw the fastener (retrograde motion) through a tunnel in a bone. Further, putting the driver into tension to assist in the drawing (or withdrawal) of the fastener through a tunnel cannot disengage the driver from the fastener. A small clockwise rotation of the driver realigns the pins with the channels so that the tip can be withdrawn from the fastener.

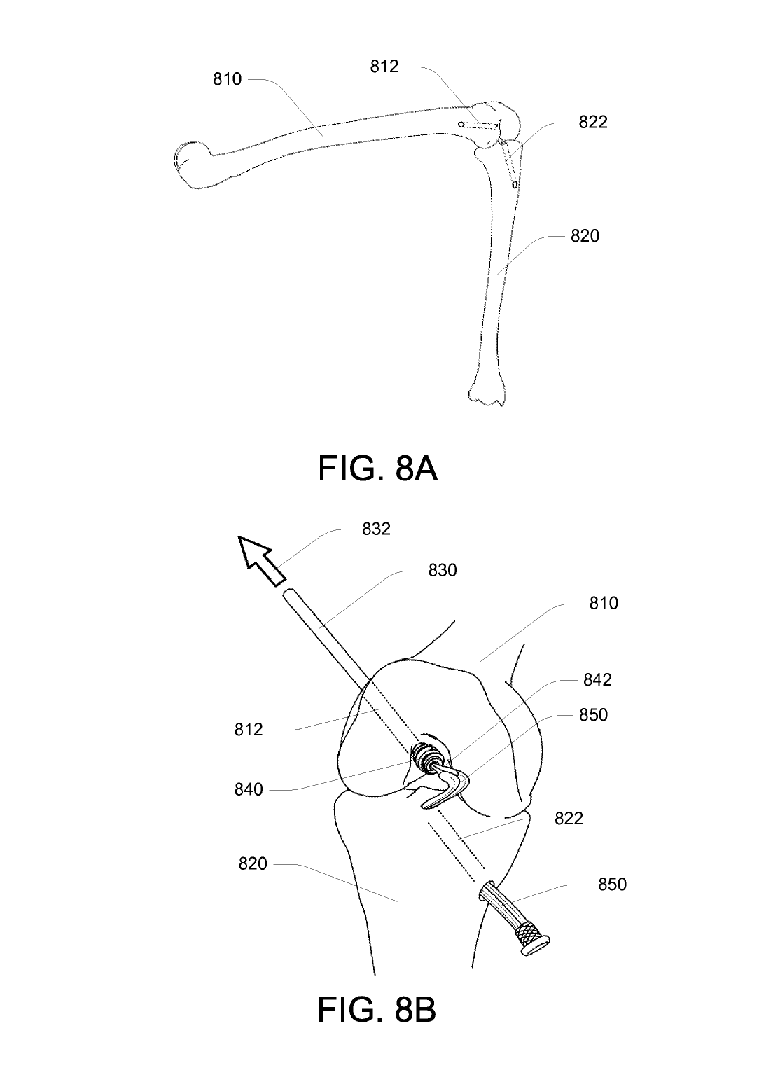

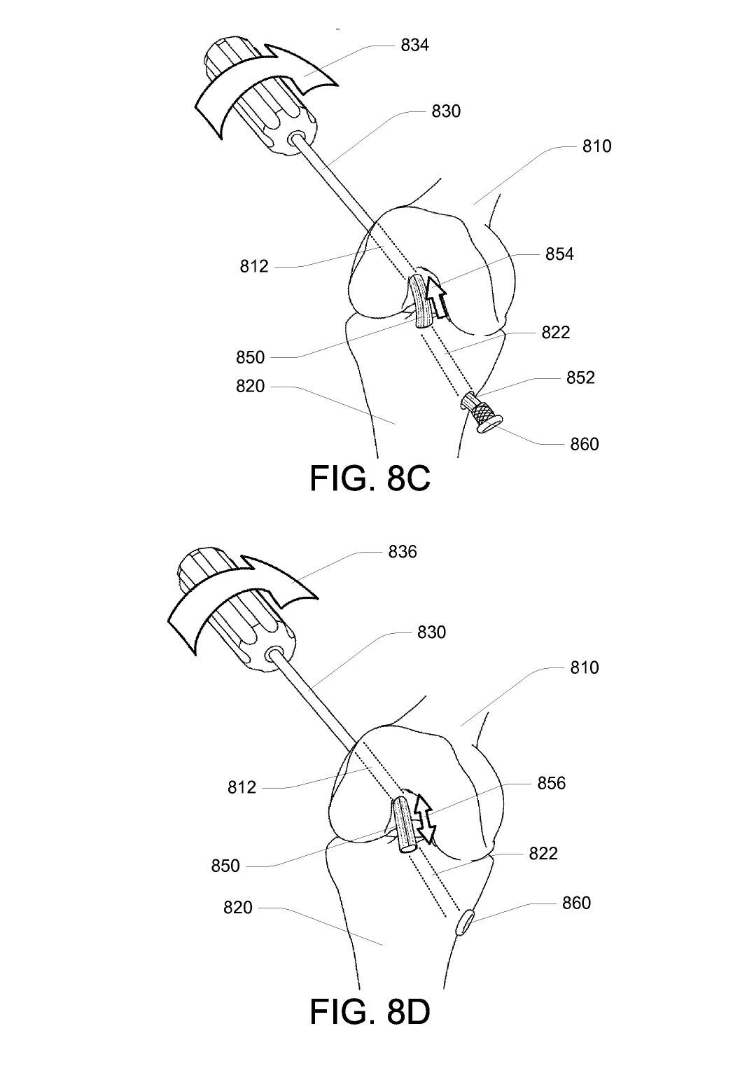

Referring to FIG. 8A through 8D, a method of using a fastener in an ACL reconstruction is disclosed.

FIG. 8A shows holes are first drilled in the femur 810 and tibia 820 to form a femoral tunnel 812 and tibial tunnel 822.

FIG. 8B shows that a driver 830 can be passed through either tunnel (1512 or 822), in any direction. The screw fastener 840 can be prefixed on the driver or placed in position once the driver is passed through the holes. The tendons/graft 850 can be rotatably coupled to the screw fastener 840, for example by passing the tendons/graft through a loop 842 located about a saddle (not shown). The driver can then be pulled up through the tunnel (as indicated by arrow 832) such that the screw fastener engages the bone.

FIG. 8C shows that as the driver 830 and coupled screw fastener (not shown) can be rotated (as indicated by arrow 834) with respect to the respective bone. This causes the screw fastener to threadedly engage the bone and thereby draw the tendons/graft 850 up through the tunnel (as indicated by arrow 854). In this embodiment, the saddle rotates within the screw as the graft is being pulled up so as to not twist the graft. The opposite end of the graft 852 is fixed relative to the tibia 820. By way of example only, an end plug 860 can be fixed opposite end of the graft 852, such that drawing up the graft brings the end plug 860 into abutting engagement (or seated) with the tibia 820. It will be appreciated that the opposite end 852 of the tendons/graft 850 to the screw (or the tendons/graft free end) can be fixed in any surgically suitable manner.

FIG. 8D shows that once the opposite end 852 of the graft is fixed relative to the tibia 820, the driver 830 and coupled screw fastener (not shown) can be rotated in a clockwise or anticlockwise direction with respect to the respective bone (as indicated by arrow 836). This causes the screw fastener to threadedly engage the bone and thereby increases or decreases tension applied to tendons/graft 850 (as indicated by arrow 856). A torque driver can be used to fine tune the tension applied to the tendons/graft 850.

The driver 830 can then be detached from the screw fastener 840.

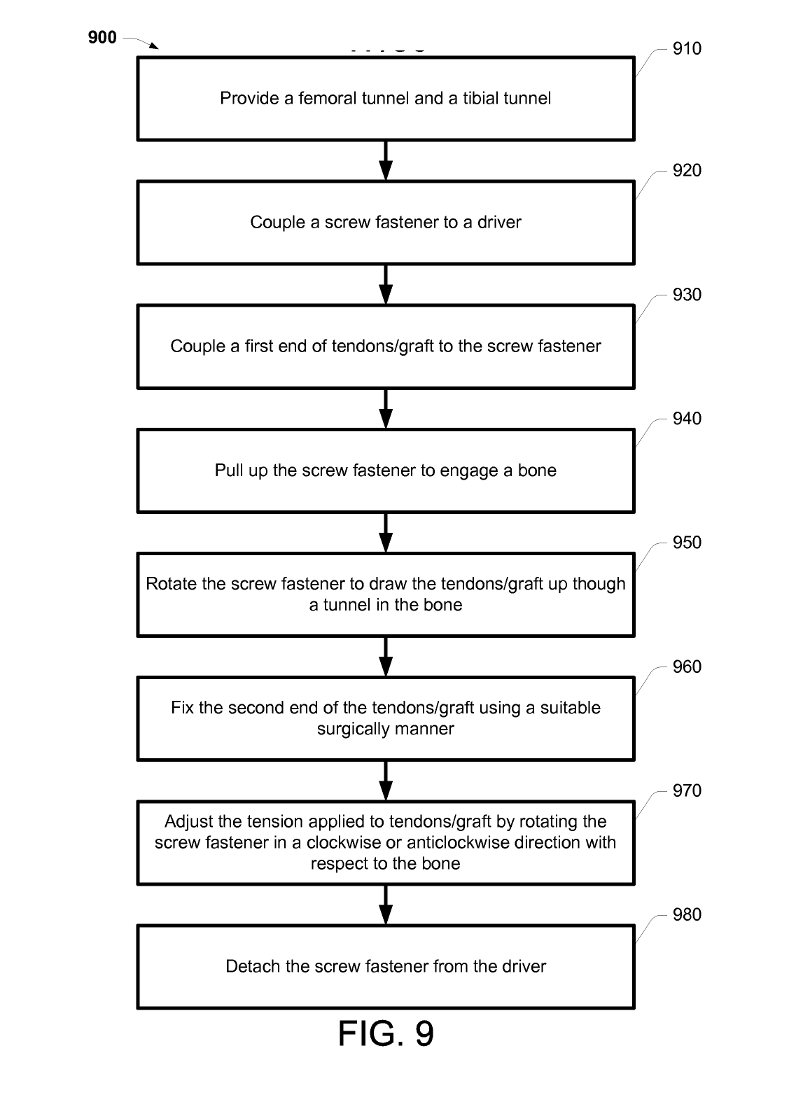

Referring to FIG. 9, a method 900 of using a fastener in an ACL reconstruction can comprise the steps of:
STEP 910: providing a femoral tunnel and a tibial tunnel in the femur and tibia respectively;
STEP 920: coupling a screw fastener to a driver passed through either tunnel;
STEP 930: rotatably coupling a graft to the screw fastener;
STEP 940: pulling up the screw fastener engages a bone;
STEP 950: rotating the screw fastener, by rotating the driver, causing the screw fastener to threadedly engage the bone and thereby draw the graft up through the tunnel.
STEP 960: fixing the opposite end of the graft to the screw (or the tendons/graft free end) to a bone using a suitable surgical manner;
STEP 970: with the opposite end of the graft fixed relative to a bone, the screw fastener (and coupled driver) can be rotated in a clockwise or anticlockwise direction with respect to a respective bone to thereby increase or decrease tension applied to tendons/graft.
STEP 980: detaching the screw fastener from the driver.

In this embodiment, a saddle rotates within the screw as the tendons/graft is being pulled up so as to not twist the graft. The opposite end of the graft can be fixed relative to the tibia. By way of example only, an end plug can be fixed to the opposite end of the graft, such that drawing up the graft brings the end plug into abutting engagement (or seated) with the tibia. It will be appreciated that the opposite end of the graft to the screw (or the tendons/graft free end) can be fixed in any surgically suitable manner.

It will be appreciated that a graft (for example a ligament or tendon, being original, transplanted or artificial) used in surgery is typically fixed in place once in the final position. Typically a screw, or similar device, is inserted into a tunnel in bone housing the graft and used to squash the graft against the bone. In this way an interference fit is formed. Alternatively, a graft can be sutured and tied onto a fixation device located outside of the bone tunnel.

In an embodiment, a retainer device can be fixed onto the graft before they are drawn to (or pulled into) their final position. This device is fixed (possible releasably) onto a graft (for example a ligament or tendon, being original, transplanted or artificial) to restrict the graft from being drawn up through a bone tunnel.

It will be appreciated that a retainer device can be used in an anterior cruciate ligament reconstruction, but is not limited to this particular field of use. Preferably, the retainer device can used in conjunction with a screw fastener device that pulls a ligament into a hole in bone.

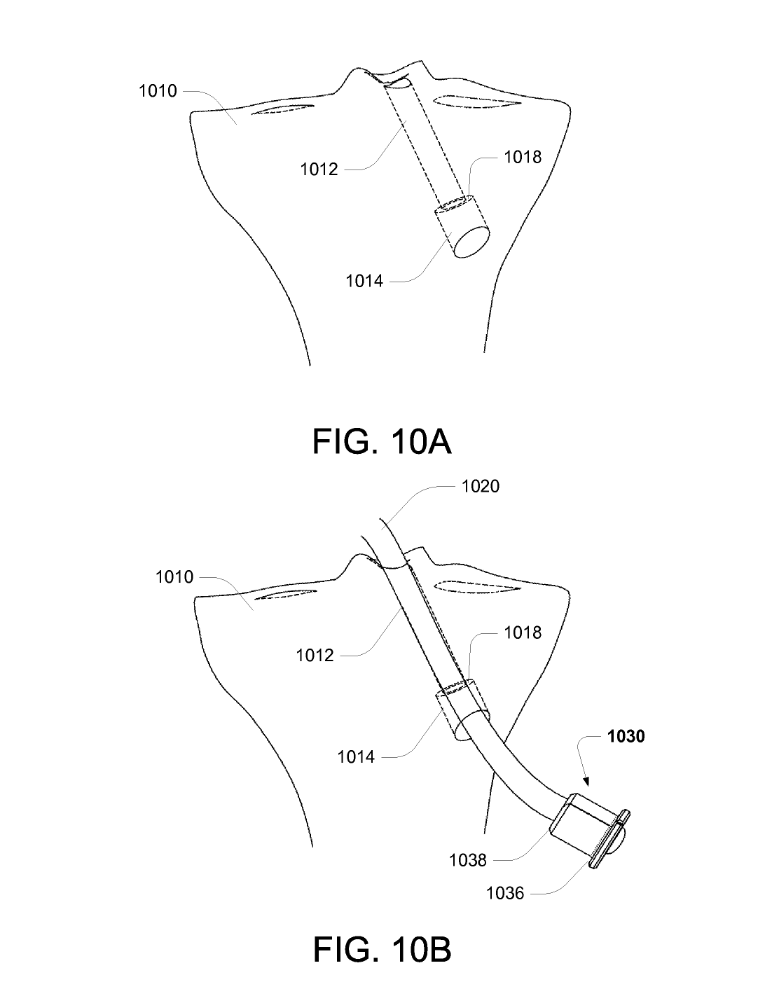

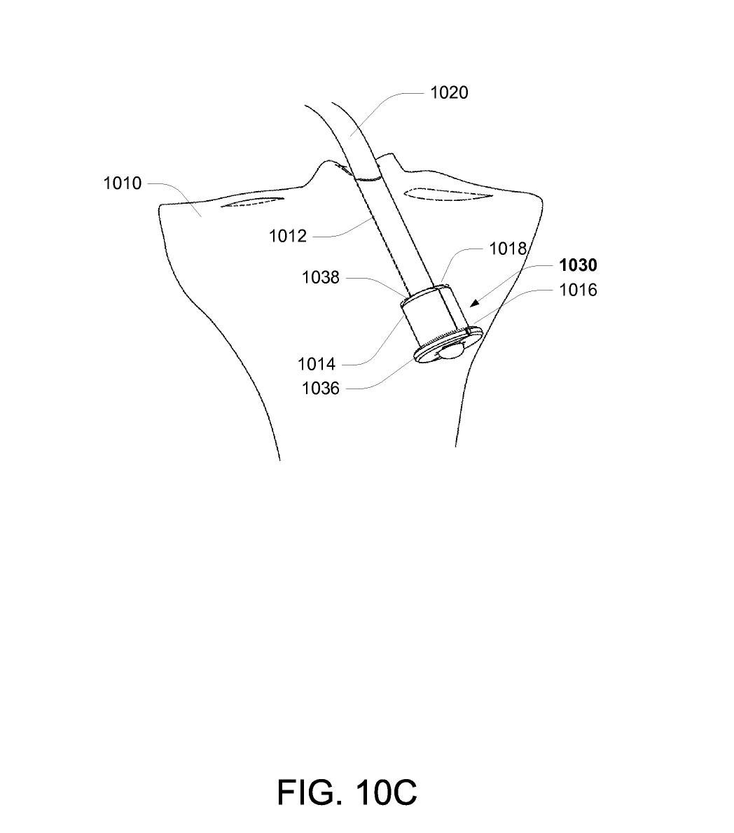

FIG. 10A-10C shows perspective views illustrating steps involved in utilisation of an embodiment graft retainer device in conjunction with an ACL reconstruction.

Referring to FIG. 10A, a bone 1010 is first drilled to form a tunnel 1012. An expanded aperture 1014 at the surface of the bone can be provided to enable a retainer device 1030 to abut a circumferential surface 1018 defined by the expanded aperture. This can enable axial alignment of the device when drawn in place, and/or further enable the device to be drawn into the bone to thereby minimise or reduce protrusion of the device above the surface of the bone (as best shown in FIG. 10C).

Referring to FIG. 10B, a graft 1020 is provided within the bone tunnel 1012. The graft retainer device 1030 can be fixed (for example clamped) to the graft before being drawn (or pulled) into position. For example, graft retainer device 1030 can be fixed to the graft before being inserted into the hole or prior to being drawn (or pulled) into position.

With the retainer device 1030 fixed to the graft, the graft is further drawn (or pulled) into position such that retainer device abuts the bone, thereby stopping progress into the bone.

The graft can then be tensioned from the other end. Preferably, tension can be applied to the graft by use of a screw fastener device pulling a graft. It will be appreciated that, when used in combination with a screw fastener device, the graft is not significantly rotated (or twisted) as it is pulled or tensioned, and the retainer device stops progress into the bone.

It will be appreciated that progress of a graft having a fixed retainer device 1030 can be restricted by a plurality of restriction means.

It will be appreciated that progress of the graft can be restricted by forming an expanded abutment surface at the end of the graft. This abutment surface is typically provided by a circumferential flange 1036 on the retainer device 1030, but can be formed in any shape or provided in the form of a plurality of radial protrusions. The abutment surface abuts—directly or indirectly—against the outside of the bone 1016 or within the bone tunnel 1012. The progression of the graft can also be restricted by a base surface 1038 of the retainer device 1030 abuttingly engaging the circumferential surface 1018 defined by the expanded countersunk aperture.

It will be further appreciated that progress of the graft can be restricted by forming a conical abutment surface at the end of the graft. This conical abutment surface is typically provided by configuring the external surface of the retainer device 1030 to define a frusto-conical shape. An interference fit can be established between the frusto-conical abutment surface and the bone tunnel or a cooperating conical recessed formed proximal to the surface of the bone.

A result of the above configurations is that a graft can be fixed such that movement of the graft further into the bone tunnel is resisted, thereby enabling the graft to be tensioned.

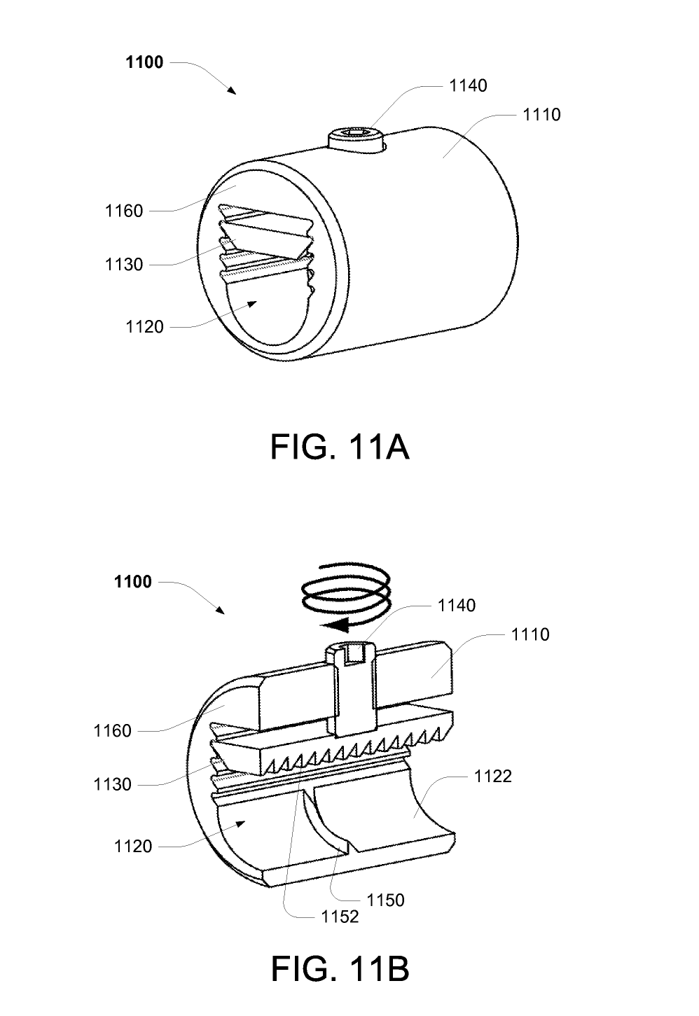

FIG. 11A and FIG. 11B shows an embodiment graft retainer device 1100. This retainer device 1100 has a body 1110 defining an aperture 1120 there through. A moveable inner gripping element 1130, in the form of a moveable elongate element, is provided within the aperture.

With a graft inserted through the aperture (not shown), the elongate element can be moved to press the graft between the elongate element and an inner surface 1122 of the body 1120.

A grub screw 1140 can be used to apply downward (with reference to the drawings) force to the elongate element 1130, for pressing the graft between the elongate element and an inner surface 1122 of the body 1120.

Gripping protrusions (for example 1150 and 1152) can be applied to surfaces that are adapted to contact a graft, thereby providing further facilitating fixing engagement between the retainer device 1100 to the graft (not shown). In this embodiment, the retainer device 1100 is releasably fixedly engageable to the graft (not shown).

The retainer device 1100 has an abutment surface 1160 being one end of the body. It will be appreciated that progress of the graft can be restricted by forming an abutting engagement—directly or indirectly—between the abutment surface 1160 and a bone. It would be appreciated that, as the abutment surface 1160 the retainer device 1100 is the leading edge, a counter sunk hole is preferred to enable the device to traverse (at least partially) into the bone before forming an abutting engagement.

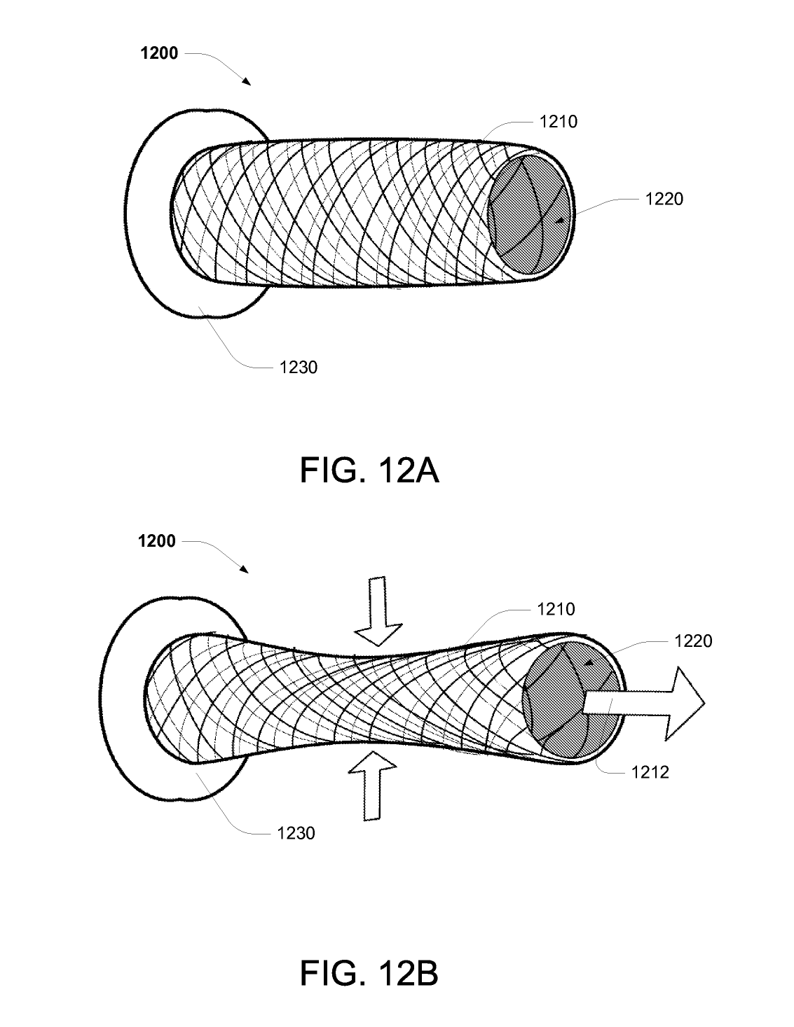

FIG. 12A and FIG. 12B shows an embodiment graft retainer device 1200. This retainer device 1200 has a body 1210 defining an aperture 1220 there through. In this embodiment, the body 1210 is defined by a woven suture element (similar in structure to a Chinese finger trap).

In this embodiment, with a graft (not shown) is inserted through the aperture, the structure of the body resists axial movement 1212 of the graft by exerting a reactive inward radial force 1214 to establish a fixing engagement between the retainer device 1200 to the graft (not shown). The retainer device 1200 is releasably fixedly engageable to the graft (not shown).

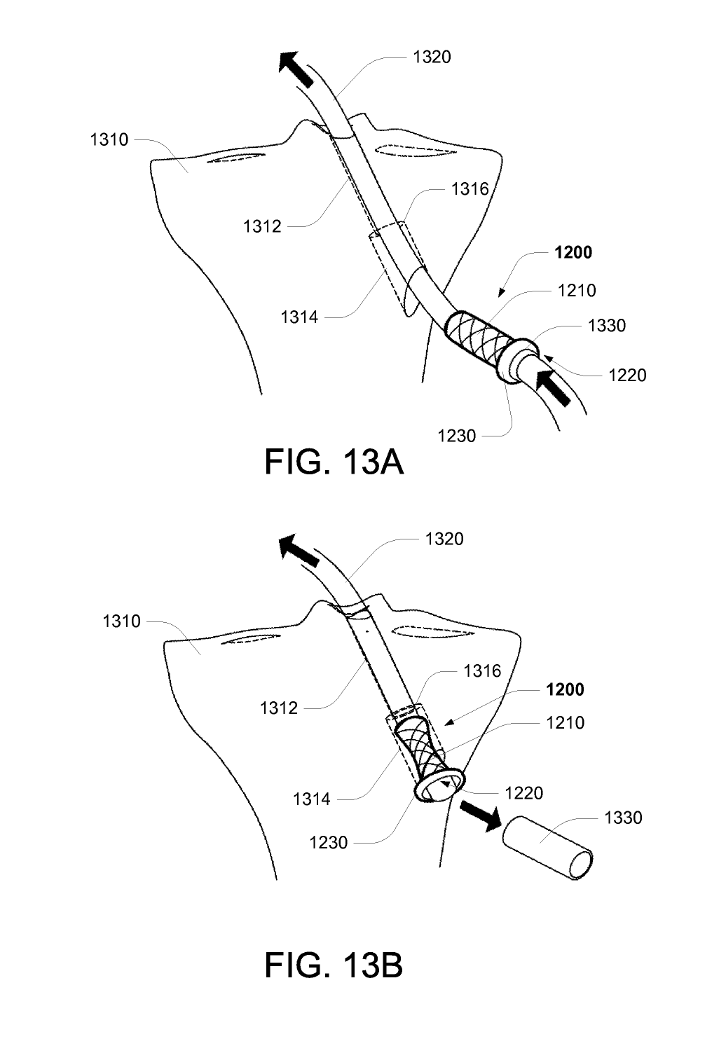

The retainer device 1200 can further include a circumferential flange 1230 abutment surface. It will be appreciated that progress of the graft can be restricted by forming an abutting engagement—directly or indirectly—with the bone (as best shown in FIG. 13B).

FIG. 13A and FIG. 13B show steps of a method of using a graft retainer device 1200. A cylindrical inner sleeve 1330 can be provided (or inserted) within the aperture 1220 of the body 1210. This enables a graft to be inserted through the aperture and the retainer device 1200 positioned along the graft to a selected position.

Referring to FIG. 13B, the inner sleeve can be removed to enable the structure defined by the body 1210 to fixedly engage the graft and resist relative movement there between.

As the graft 1320, and fixedly engaged graft retainer device 1200, is further drawn through the bone tunnel 1312, the circumferential flange 1230 abutment surface comes into abutting engagement—directly or indirectly—with the bone, thereby restricting further progress of the graft through the tunnel.

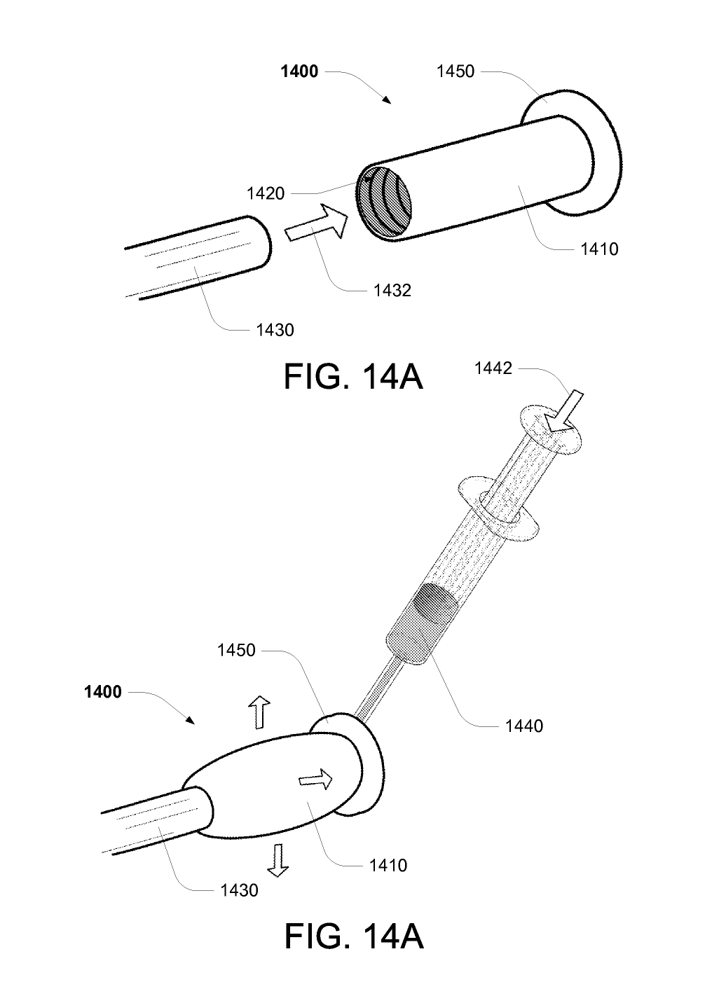

FIG. 14A and FIG. 14B shows an embodiment graft retainer device 1400. This retainer device 1400 has a body 1410 defining an aperture 1420 there in (or there through). In this embodiment, the body 1410 is defined by a substantially cylindrical sleeve.

The graft retainer device 1400, in use, enables a graft 1430 to be inserted into the aperture (as in indicated by arrow 1432). The aperture defines an axial direction for insertion of the graft (and typically defining a direction of tension applied to the graft in use).

A fluid agent 1440 can be inserted 1442 into the body 1410, as best shown in FIG. 14B. The agent enables the body to resist respective axial movement 1212 with the graft. By way of example only, a reactive inward radial force is exerted by the inserted fluid agent. Alternatively, the fluid agent can fixedly engage the body 1410 to the graft 1430.

The retainer device 1400 can further include a circumferential flange 1450 abutment surface. It will be appreciated that progress of the graft can be restricted by forming an abutting engagement—directly or indirectly—with the bone.

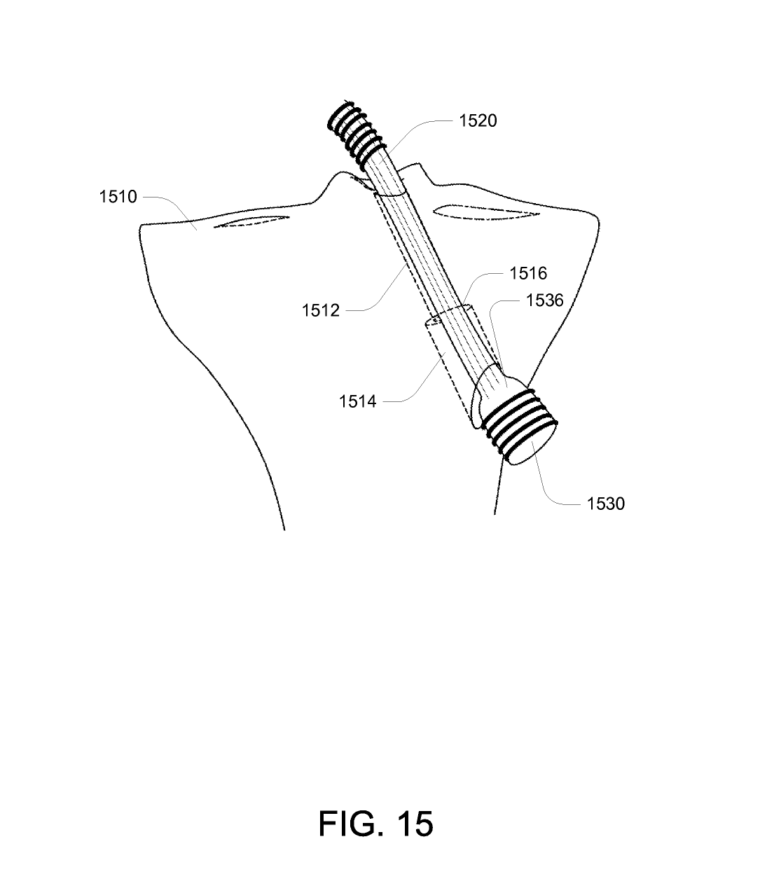

FIG. 15A shows a perspective view of an embodiment artificial graft 1520 having an integrally formed graft retainer device 1530.

In this embodiment the retainer device 1530 is defined by a broadening of the artificial graft, thereby forming an abutment surface.

In use, a bone 1510 is first drilled to form a tunnel 1512. An expanded aperture 1514 at the surface of the bone can be provided to define a circumferential surface 1516. As the graft 1520 is drawn (or pulled) through the tunnel 1512, the abutment surface 1536 of the retainer device 1530 can come into abutting engagement with the circumferential surface 1516. It will be appreciated that progress of the graft can be restricted by forming an abutting engagement—directly or indirectly—with the bone.

This can enable axial alignment of the device, graft and tunnel when drawn in place, and/or further enable the device to be drawn into the bone to thereby minimise or reduce protrusion of the device above the surface of the bone.

In other configurations, the tendons could also be held with sutures, wherein the ends of the sutures could finish in a loop through which a graft retainer device can be inserted to thereby restrict the graft travelling through the bone tunnel.

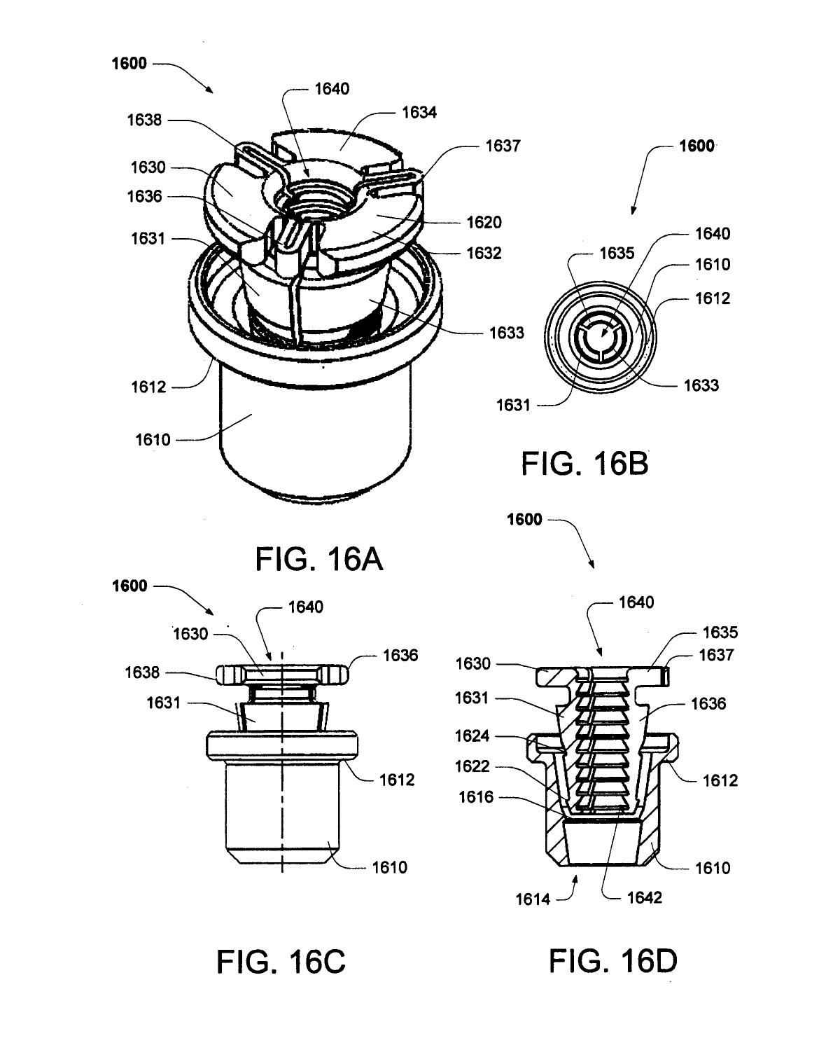

FIG. 16A through FIG. 16D show an embodiment graft retainer device 1600.

This embodiment comprises a body 1610 having a circumferential flange 1612 abutment surface, and defining an aperture 1614 there in (or there through). In this embodiment, the body 1610 is defined by a substantially cylindrical sleeve. The aperture defines an axial direction for insertion of the graft (and typically defining a direction of tension applied to the graft in use).

An insert component 1620 includes three resilient interconnected elements 1630, 1632 and 1634 each having a respective finger protrusion 1631, 1633 and 1636. The elements 1630, 1632 and 1634 are interconnected about a circumference by resilient hinge members 1636, 1337 and 1638. This resilient connection enables the finger protrusions to move radially inwardly and outwardly in use. The interconnected finger protrusions define an aperture 1640.

The graft retainer device 1600, in use, enables a graft (not shown) to be inserted into (and through) the aperture 1640. Inserting the insert component 1620 into the body aperture 1614 brings the finger protrusions into sliding abutting engagement with an internal sidewall 1615 defining the aperture 1614. The sidewalls configured to neck down, thereby restricting the aperture 1614 and thereby causing the finger protrusion to move radially inward into a clamping engagement with the graft. In this embodiment the finger protrusions are also configured to be outwardly broadening as extending from the distal insertion end of the insert component 1620.

It would be appreciated that alternative embodiments may include either the insert component 1620 and/or the aperture 1614 being adapted to cause clamping engagement between the finger protrusions and a graft.

Gripping formations can be included along the aperture 1640, for providing improved graft retention.

The body 1610 and the insert component 1620 may further include one or more cooperating locking elements for locking the insert element relative to the body 1610 when in use. In this embodiment, the body aperture 1614 includes a inwardly directed circumferential locking element in the form of a flange 1616 which is operatively associated with outwardly directed flange 1622 on each finger protrusion—thereby adapted to form an abutting locking engagement there between, while the finger protrusion are in clamping engagement with a graft. It will be appreciated that the device can be adapted to have a plurality of lockable positions (for example using 1622 and/or 1624)

The retainer device 1600 includes a circumferential flange 1612 abutment surface. It will be appreciated that progress of the graft can be restricted by forming an abutting engagement—directly or indirectly—with the bone.

It will be appreciated that the body 1610 and insert component 1620 can be threadedly engaged such that relative rotation therebetween axially moves the insert component with respect to the body, thereby clamping or unclamping a tendon.

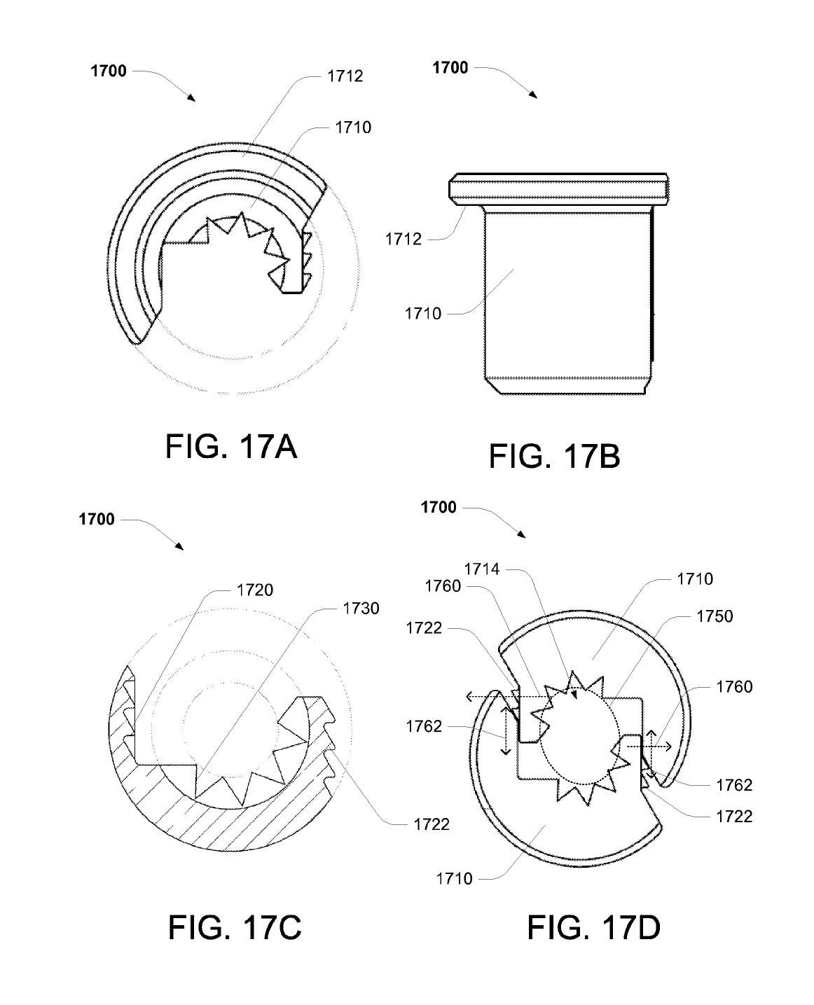

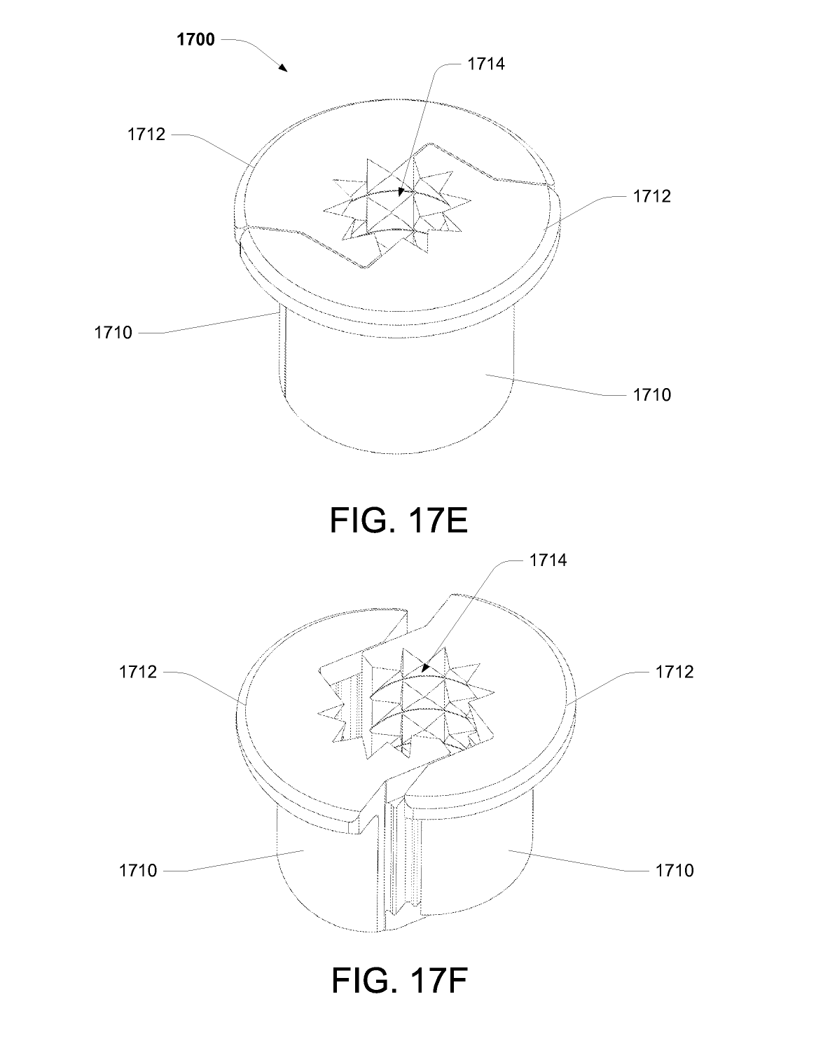

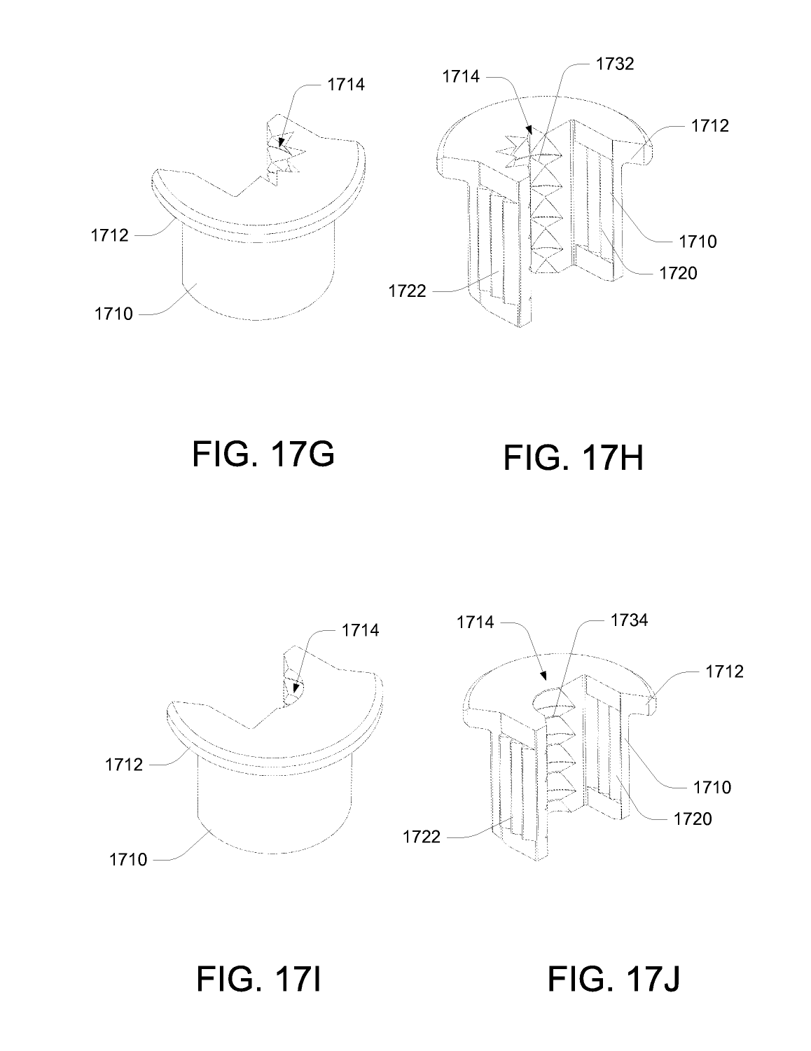

FIG. 17A through FIG. 17D show an embodiment graft retainer device 1700. This embodiment comprises two body elements 1710 (as best shown in FIG. 17D) having a circumferential flange 1712 abutment surface, and defining an aperture 1714 there in (or there through).

In this embodiment, each body element 1710 defines a complimenting portion of a cylindrical sleeve. The aperture defines an axial direction for insertion of the graft (and typically defining a direction of tension applied to the graft in use).

Respective cooperating locking elements 1720 and 1722 are included to lock the two body element 1710 in clamping engagement with a graft 1750 located within the aperture 1714. In this example the locking elements 1720 and 1722 are cooperating ratchet like protrusions adapted to restrict separation of the body elements 1710 in one direction.

Referring to FIG. 17D, it will be appreciated that in this embodiment, the graft further cooperates with the engaged locking elements 1720 and 1722 by restricting respective movement between the body elements—providing a retaining force (as indicated by arrows 1760) substantially perpendicular to the movement restriction provided by the locking elements (as indicated by arrows 1762)—thereby restricting disengagement of the coupled body elements.

Gripping formations 1730 can be included along the aperture 1714, for providing improved graft retention.

Referring to FIG. 17E and FIG. 17F, it will be appreciated that in this embodiment, the graft further cooperates with the engaged locking elements 1720 and 1722 by restricting respective movement between the body elements—providing a retaining force substantially perpendicular to the movement restriction provided by the locking elements—thereby restricting disengagement of the coupled body elements.

It will be appreciated that a respective pair of cooperating body elements 1710 can be in clamping engagement with a graft located within the aperture 1714, while in either a completely closed clamping configuration (as best shown in FIG. 17E) or a substantially closed clamping configuration (as best shown in FIG. 17F).

Referring to FIG. 17G through FIG. 17J, gripping formations can be included along the aperture 1714, for providing improved graft retention. These gripping formations, by way of example, can include spiked or localised protrusions 1732 (as best shown in FIG. 17H) or can form circumferential rigged protrusions 1734 (as best shown in FIG. 17J).

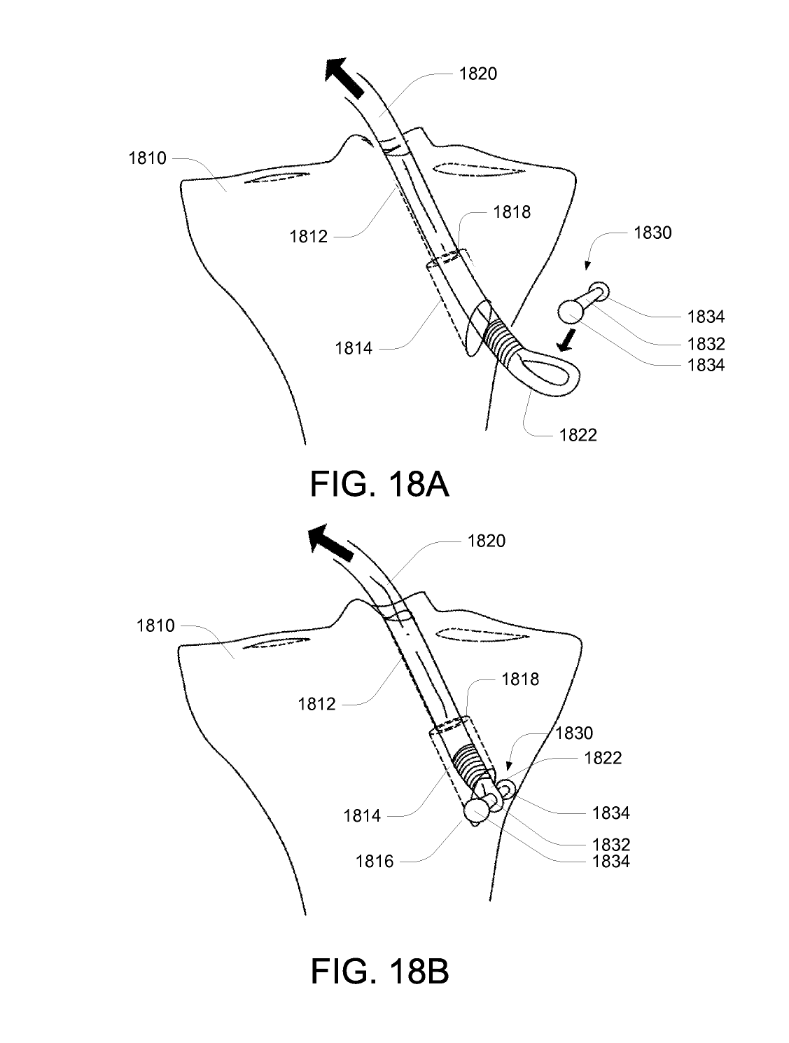

Referring to FIG. 18A, a bone 1810 is first drilled to form a tunnel 1812. An expanded aperture 1814 at the surface of the bone can be provided to enable a graft 1820 having a looped end 1822 enter the bone. This can enable the graft retainer device 1030 to be drawn against the bone to thereby minimise or reduce protrusion of the device above the surface of the bone (as best shown in FIG. 18B).

Referring to FIG. 18A and FIG. 18B, a graft 1820 is provided within the bone tunnel 1812. The graft retainer device 1830 can be fixed, by placing the device through a loop 1822 formed at the end of the graft before being drawn (or pulled) into position. For example, graft retainer device 1830 can be fixed to the graft before being inserted into the hole or prior to being drawn (or pulled) into position.

With the retainer device 1830 fixed to the graft, the graft is further drawn (or pulled) into position such that retainer device abuts the bone at 1816, thereby stopping progress into the bone.

The graft can then be tensioned from the other end. Preferably, tension can be applied to the graft by use of a screw fastener device pulling the graft. It will be appreciated that, when used in combination with a screw fastener device, the graft is not significantly rotated (or twisted) as it is pulled or tensioned, and the retainer device stops progress into the bone.

It will be appreciated that the retainer device 1830 can comprise a shaft 1832 having enlarged ends portions 1834, thereby defining a dumb-bell shape. The loop 1822 formed at the end of the graft can be placed over the enlarged ends portions 1834 and be retained by the shaft. A retainer device 1830 can also be formed in other shapes that provide equivalent retention of the graft loop.

It will be further appreciated that the retainer device 1830 can be used in combination with a clamping device (for example 1700, 1600, 1400, 1200, 1100, 1030) when the clamping device further comprises a loop for receiving the retainer device 1830.

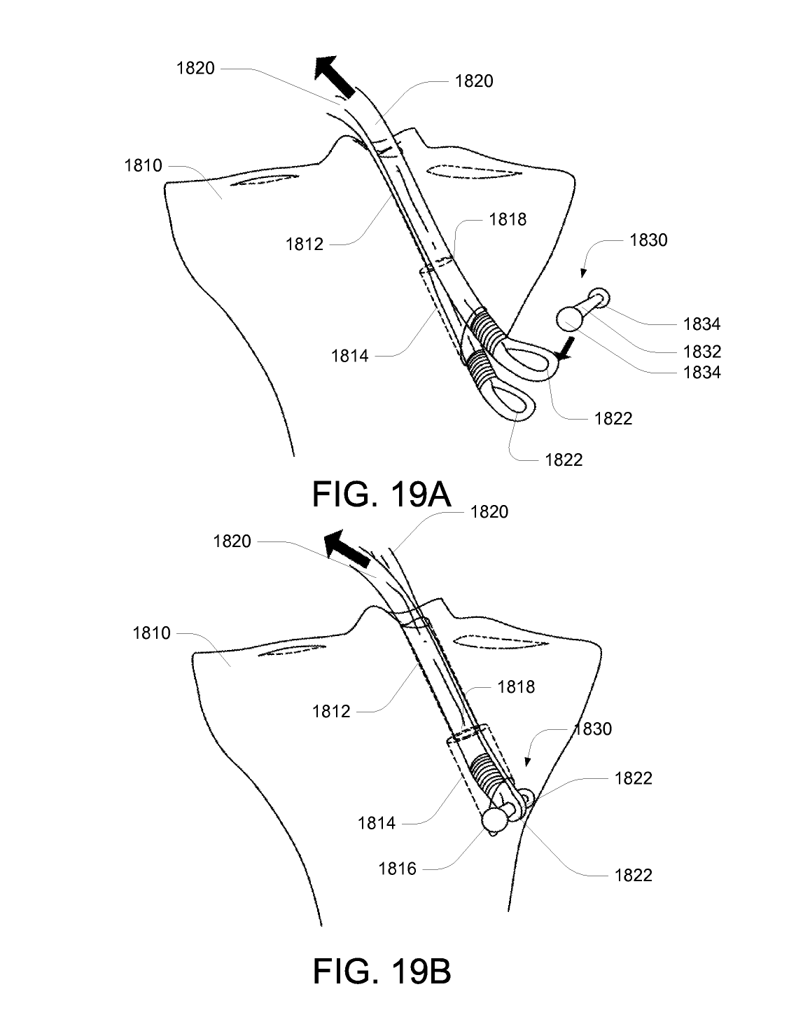

It will be further appreciated that the retainer device 1830 can be used to retain a plurality of grafts, as best shown in FIG. 19A and FIG. 19B.

A result of the above configurations is that a graft can be fixed such that movement of the graft further into the bone tunnel is resisted, thereby enabling the graft to be tensioned.

It will appreciated that a retainer device can further include an abutment surface (for example a circumferential flange), in many forms. It will be appreciated that progress of the graft can be restricted by forming an abutting engagement—directly or indirectly—between the device and the bone. A graft retainer device can abut against the outside or outside of a bone or form an interference fit therebetween.

It will be appreciated that for each embodiment graft retainer device a graft can be retained with respect to a bone so that it cannot be pulled further into a bone tunnel. The device itself can be of multiple configurations, for example, it can comprise a single piece or a plurality of pieces.

A graft retainer device can comprise a two piece configuration, with an outer threaded portion applying force to an inner portion, wherein the inner portion can be screwed/forced down for clamping a graft against the inner wall of the device.

A graft retainer device can include protrusions or roughening of an internal wall defining an aperture, for improved gripping engagement with a graft. Protrusions can include spikes, raised edges or any other shape that will push into the body of a graft. Alternatively, an embodiment can include an outer sleeve through which a graft is inserted, and an inner portion adapted to push the graft against the walls of outer sleeve.

By way of example only, using a suture type device could vary depending on the number of tendons. These grafts can also be attached to each other so they are on the same place on each tendon. The graft retainer device can be passed over the tendons, preferably using a cylindrical insertion device which passes over the grafts. This could be done either as the grafts are being prepared on a separate table or as they are about to be passed into the tibial or femoral tunnel. The device grabbing the grafts may be a separate suture that can be pulled into atype of slip knot arrangement such that the device can then be pulled back leaving the device on the tendons in a tightened position. The other end of the device is then fixed to a bone in a number of ways. One typical way would be for there to be a loop at the end of the device or if in 2 parts they could be connected by a loop or band of the weave and a separate device such as a cross bar, cylinder, screw used to stop the tendon progressing into the bone as the other end is tensioned.

By way of example only, using an inflatable membrane into which a fluid material (including any artificial bone) could be injected to compress the graft, thereby retaining the graft therein. This technique can used once the tendons have been seated.

All of these graft retainer devices can be placed on the ligament before it is seated or pulled into the tunnel. This can be performed once the ligament has been placed in the tunnel (hole) or it can be done on a separate work bench before implantation. Preferably, the other end of the graft is operatively associated with a tensioning device.

It will be appreciated that a graft retainer device can comprise one or more of a plurality of materials, preferably bio-absorbable polymer.

Preferably, when using a graft retainer device, the respective bone tunnel is expanded to the outer diameter and the shape of device before implantation. Alternatively, the same diameter bone tunnel may be used, for example when using a suture technique, such that abutment against the outside of the bone restricts the graft from being drawn further into the tunnel.

According to an embodiment, there is provided graft retainer device that can be placed on a graft before being placed into a bone. The device can comprise a single piece construction or a plurality of components, which can clamp around the graft by opening up sideways or longitudinally.

While the present invention has been disclosed with reference to particular details of construction, these should be understood as having been provided by way of example and not as limitations to the scope or spirit of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

Similarly, it is to be noticed that the term coupled, when used in the claims, should not be interpreted as being limitative to direct connections only. The terms “coupled” and “connected”, along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical, or that two or more elements are not in direct contact with each other but yet still co-operate or interact with each other.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

As used herein, unless otherwise specified the use of terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader, or with reference to the orientation of the structure during nominal use, as appropriate. Similarly, the terms “inwardly” and “outwardly” generally refer to the orientation of a surface relative to its axis of elongation, or axis of rotation, as appropriate.

Similarly it should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

It will be appreciated that an embodiment of the invention can consist essentially of features disclosed herein. Alternatively, an embodiment of the invention can consist of features disclosed herein. The invention illustratively disclosed herein suitably may be practiced in the absence of any element which is not specifically disclosed herein.

1. A graft retainer device for substantially retaining a portion of a graft with respect to a bone of a patient, the device comprising:
a body; and
a retaining element for retaining a graft with respect to the body.

2. The device according to claim 1, wherein the body defines an aperture for receiving a graft.

3. The device according to claim 2, wherein the retaining element is a gripping element for retaining a graft received through the aperture.

4. The device according to claim 2, wherein the body includes an abutment surface that is adapted to abut the bone for substantially fixing one portion of the graft with respect to the bone.

5. The device according to claim 2, wherein the device is drawn at least partially into a tunnel formed within the bone, thereby to reduce protrusion of the device above a surface of the bone.

6. The device according to claim 5, wherein an interference fit is established between the body and the tunnel formed within the bone.

7. The device according to claim 1, wherein the device is fixed to the graft before insertion into a tunnel defined by the bone, such that the device abuttingly engages the bone before the graft is tensioned.

8. The device according to claim 1, wherein the retaining element includes a moveable inner gripping element, the inner gripping element and body define an aperture for receiving the graft, such that movement of the gripping element is adapted to retain the graft with respect to the body.

9. The device according to claim 1, wherein:
the retaining element includes an insert element that is insertable in a through aperture defined by the body;
the insert element including a plurality of interconnected elements, each having a respective finger protrusion; and
the finger protrusions define an aperture for receiving the graft, and are adapted to move radially inwardly to retain the graft with respect to the body.

10. The device according to claim 1, wherein the retaining element includes a woven suture element that defines an aperture for receiving the graft and is adapted to retains the graft with respect to the body.

11. The device according to claim 1, wherein the gripping element defines an aperture for receiving the graft and includes a fluid agent inserted into the body for retaining the graft with respect to the body.

12. The device according to claim 1, wherein:
the body comprises at least two body elements;
the body elements, when assembled, define the aperture for receiving a graft.

13. The device according to claim 8, wherein the aperture for receiving the graft includes inwardly directed gripping protrusions.

14. The device according to claim 1, wherein the device is integrally formed with an artificial graft.

15. (canceled)

16. The device according to claim 1, wherein the device is used with a surgical screw fastener that pulls the graft through a first tunnel defined in the bone, the surgical screw fastener including:
a fastener body having a proximal end and a distal end;
an exterior screw thread located around the fastener body for threadedly engaging a wall of the first tunnel;
a first coupling element at the proximal end of the fastener body, the first coupling element adapted to couple a driver tool; and
a second coupling element for rotatable coupling a first end of the graft with respect to the fastener.

17. A surgical screw fastener for pulling a graft through a tunnel defined in a bone, the graft being retained by a graft retainer device according to claim 1.

18. The surgical screw according to claim 17, wherein the fastener includes:
a fastener body having a proximal end and a distal end;
an exterior screw thread located around the fastener body for threadedly engaging a wall of the first tunnel;
a first coupling element at the proximal end of the fastener body, the first coupling element adapted to couple a driver tool; and
a second coupling element for rotatable coupling a first end of the graft with respect to the fastener.

19. A method of substantially retaining a portion of a graft with respect to a bone of a patient, while using a surgical screw fastener to pull the graft through a tunnel defined in the bone, the method comprising the steps of:
(a) providing a graft retainer device according to claim 1;
(b) fixing the graft retainer device to a first portion of the graft;
(c) providing a screw fastener;
(d) coupling the screw fastener to a driver tool passed through the first tunnel;
(e) rotatably coupling a second portion of the graft to the screw fastener;
(f) rotating the screw fastener, by rotating the driver tool, causing the screw fastener to threadedly engage the bone and thereby draw the graft up through the tunnel;
(g) with the first portion of the graft retained, the screw fastener can be rotated with respect to the bone to thereby set a tension applied to the graft; and
(h) detaching the driver tool from the screw fastener.

20. (canceled)
